(19) United States
(12) Patent Application Publication
(10) Pub. No.: US 2018/0228158 A1
(43) Pub. Date: Aug. 16, 2018

(54) COMPOSITIONS AND METHODS FOR CONTROL OF DISEASE

(71) Applicant: Monsanto Technology LLC, St. Louis, MO (US)

(72) Inventors: Barry J. Shortt, New Melle, MO (US); Michael S. South, Chesterfield, MO (US); Al S. Wideman, St. Louis, MO (US); Deryck Jeremy Williams, University City, MO (US)

(73) Assignee: Monsanto Technology LLC, St. Louis, MO (US)

(21) Appl. No.: 15/890,948

(22) Filed: Feb. 7, 2018

Related U.S. Application Data
(60) Provisional application No. 62/455,906, filed on Feb. 7, 2017.

(51) Int. Cl. A01N 43/82; C07D 413/04

(52) U.S. Cl. CPC A01N 43/82; C07D 413/04

(57) ABSTRACT

Compounds, compositions and methods for controlling root-originating diseases are described herein. The compounds include oxazoles, oxadiazoles and thiadiazoles. The compounds may be administered to plants, seeds, and/or soil.

### REFERENCE TO RELATED APPLICATIONS

This application claims the benefit of U.S. Provisional Application Ser. No. 62/455,906, filed Feb. 7, 2017, the entire contents of which are incorporated herein by reference.

### FIELD

Provided herein are methods, compounds, and compositions that are useful for the control of root-originated diseases, such as soybean sudden death syndrome caused by fungi of the genus Fusarium.

### BACKGROUND

Corn plants are susceptible to a number of root-originated diseases caused by fungal pathogens. One example is Fusarium root rot (caused by F. oxysporum, F. solani, F. verticilliodes, F. subglutinans, F. acuminatum, and F. equiseti). Other examples of root-orignated diseases in corn include seedling blight (caused by Fusarium spp., Pythium spp. and Rhizoctonia spp.) and stalk rot (caused by fungi of the genus Stenocarpella (Diplodia), Colletotrichum, and Gibberella (Fusarium)).

Cotton plants are also susceptible to a number of root-originated diseases caused by fungal pathogens. One example of Fusarium disease in cotton is seedling blight (Fusarium spp.). Other examples of root-orignated diseases in cotton include Verticillum wilt (V. dahliae) and Fusarium wilt (F. oxysporum).

Soybean plants are susceptible to a number of root-originated diseases caused by fungal pathogens. Examples of Fusarium diseases include but are not limited to, Fusarium root rot (caused by Fusarium spp), Fusarium seed and seedling blight, also referred to as “damping off” (caused by Fusarium spp.), Fusarium wilt (caused by Fusarium oxysporum and F. solani) and sudden death syndrome (caused by Fusarium virguliforme). Other examples of root-orignated diseases in soybean include Phytophthora root rot (caused by P. infestans) and charcoal rot (caused by Macrophomina phaseolina).

Of particular concern is the Fusarium disease soybean sudden death syndrome (SDS), which is endemic to soybean-growing regions worldwide. SDS is caused by the soil borne root pathogen Fusarium virguliforme. Presently, there are no good methods of controlling SDS, and yield loss in affected soybean crops can be significant.

The incidence and severity of SDS can be affected by the presence of soybean cyst nematodes (SCN) in the location where the soybean plants are grown. Although the exact nature of the relationship is not fully understood, it has been observed that symptoms of SDS are often more widespread and/or severe in locations having high SCN pressure.

It is therefore desirable to develop compositions and methods for controlling Fusarium diseases, such as sudden death syndrome, in soybean crops.

### SUMMARY

Provided herein is a method of controlling a root-originated disease in a plant in a location with nematode pressure, the method comprising administering to a plant, a seed, or soil a composition comprising an effective amount of a compound of Formula IV, Formula V, or a salt thereof

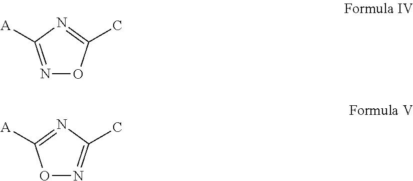

wherein A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and C is selected from the group consisting of thienyl, furanyl, oxazolyl, and isoxazolyl, each of which can be optionally independently substituted with one or more substituents selected from fluorine, chlorine, CH₃, and OCF₃.

Also provided herein is a method of controlling a Fusarium disease in a plant in a location with nematode pressure, the method comprising administering to a soybean plant, a soybean seed, or soil a composition comprising an effective amount of a compound of Formula IV, Formula V, or a salt thereof

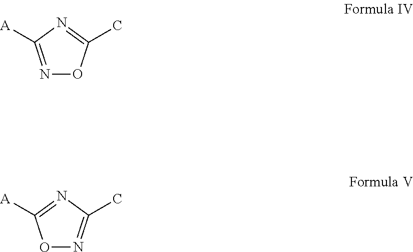

wherein A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and C is selected from the group consisting of thienyl, furanyl, oxazolyl, and isoxazolyl, each of which can be optionally independently substituted with one or more substituents selected from fluorine, chlorine, CH₃, and OCF₃.

Also provided herein is a method of controlling soybean sudden death syndrome in a plant in a location with nematode pressure, the method comprising administering to a soybean plant, a soybean seed, or soil a composition comprising an effective amount of a compound of Formula IV, Formula V, or a salt thereof

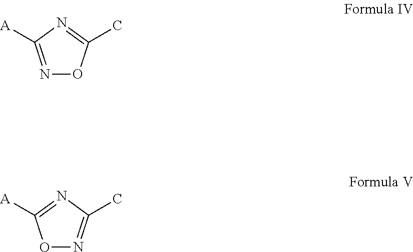

wherein A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and C is selected from the group consisting of thienyl, furanyl, oxazolyl, and isoxazolyl, each of which can be optionally independently substituted with one or more substituents selected from fluorine, chlorine, CH₃, and OCF₃.

In some embodiments of the methods provided herein, the composition is administered to a soybean plant, a soybean seed, or soil in a location wherein soybean plants are at a significant risk of disease caused by fungi of the genus Fusarium.

Also provided herein is a method of improving the yield of a soybean plant grown from a seed, the method comprising planting a soybean seed comprising a composition comprising an effective amount of a compound of Formula IV, Formula V, or a salt thereof

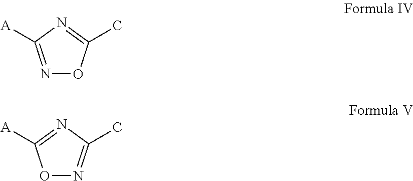

wherein A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and C is selected from the group consisting of thienyl, furanyl, oxazolyl, and isoxazolyl, each of which can be optionally independently substituted with one or more substituents selected from fluorine, chlorine, CH₃, and OCF₃.

### BRIEF DESCRIPTION OF DRAWINGS

FIG. 1 depicts the percentage of plants affected with SDS during the experimental trials described in Example 2.

FIG. 2 depicts the severity of SDS observed during the experimental trials described in Example 2.

### DETAILED DESCRIPTION

Compositions and methods for control of soybean sudden death syndrome in a plant in a location with nematode pressure are described herein.

Phytopathogenic fungi can infect agricultural crop plants, resulting in considerable economic losses to farmers and producers worldwide. Approximately 10,000 species of fungi are known to damage crops and affect quality and yield. Many fungi infect the roots of plants and result in lesions or death of part or all of the root system and are referred to herein as “root-originated disease(s).”

As used herein, the term “root-originated disease(s)” refers to fungi infecting the roots, hypocotyl or other below ground parts of plants, resulting in disease, with symptoms including lesions or death, of part or all of the root system. Non-limiting examples of fungal genuses that infect the roots of plants causing root-originated disease include Fusarium, Pythium, Phytophthora and Rhizoctonia.

As used herein, the term “Fusarium disease(s)” refer to root-originated diseases caused by fungi of the genus Fusarium that affect plants through the roots. As used herein, “controlling” Fusarium diseases broadly refers to affecting a reduction in the incidence and/or severity of the disease.

Examples of Fusarium diseases include, but are not limited to, Fusarium root rot (caused by Fusarium spp.), Fusarium seed and seedling blight, also referred to as “damping off” (caused by Fusarium spp.), Fusarium wilt (caused by Fusarium oxysporum and F. solani), and sudden death syndrome (caused by Fusarium virguliforme).

Fusarium diseases that affect plants are particularly troublesome in an agricultural context. Examples of Fusarium diseases that affect soybean plants include, but are not limited to: Fusarium root rot, Fusarium wilt, and sudden death syndrome. As used herein, the term “sudden death syndrome” and the abbreviation “SDS” each refer to the disease caused by Fusarium virguliforme that affects soybean plants. As used herein, “controlling” soybean sudden death syndrome broadly refers to affecting a reduction in the incidence and/or severity of the disease.

Examples of Fusarium diseases that affect corn plants include, but are not limited to seed rots, seedling blights, and corn stalk rot. Examples of Fusarium diseases that affect cotton plants include, but are not limited to seedling blight and Fusarium wilt.

Compounds

Described herein are compositions comprising an effective amount of a compound or a mixture of compounds having any of the formulas described herein, for example, the compounds shown below.

Described herein are compounds of Formula I or a salt thereof

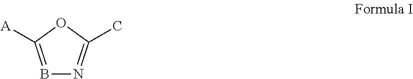

wherein,

A is phenyl, pyridyl, or pyrazyl each of which can be optionally independently substituted one or more substituents selected from: halogen, CF₃, CH₃, OCF₃, OCH₃, CN and C(H)O;

B is C(H) or C(CH₃); and

C is thienyl, furanyl, oxazolyl or isoxazolyl each of which can be optionally independently substituted with one or more substituents selected from: fluorine, chlorine, CH₃, and OCF₃.

In various embodiments: A is phenyl; A is pyridyl; A is pyrazyl; B is C(H); B is C(CH₃); C is thienyl; C is furanyl; C is oxazolyl; and C is isoxazolyl.

Also disclosed are compounds of Formula Ia or a salt thereof

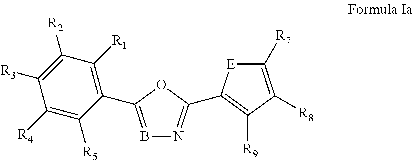

wherein,

R₁ and R₅ are independently selected from hydrogen, CH₃, F, Cl, Br, CF₃, and OCF₃,

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃;

B is C(H) or C(CH₃); and

E is O or S.

In various embodiments of the compound of Formula Ia: R₁ and R₅ are independently selected from hydrogen, fluorine and chlorine with the proviso that R₁ and R₅ cannot be simultaneously hydrogen; R₁ and R₅ are independently selected from hydrogen, chlorine and fluorine with the proviso that R₁ and R₅ cannot be simultaneously hydrogen, and R₃ is selected from Cl, Br and F; R₁ and R₅ are independently selected from hydrogen, chlorine and fluorine with the proviso that R₁ and R₅ cannot be simultaneously hydrogen and R₃ is selected from Cl and Br; R₁ and R₅ are independently selected from hydrogen, chlorine and fluorine with the proviso that R₁ and R₅ cannot be simultaneously hydrogen, R₃ is selected from Cl, Br, F, and E is S; R₁ and R₅ are independently selected from hydrogen, chlorine and fluorine with the proviso that R₁ and R₅ cannot be simultaneously hydrogen, R₃ is selected from Cl, Br, and F, E is S, and both R₂ and R₄ are hydrogen; R₁ and R₅ are independently selected from hydrogen, chlorine and fluorine with the proviso that R₁ and R₅ cannot be simultaneously hydrogen, R₃ is selected from Cl, Br, F, E is S, both R₂ and R₄ are hydrogen and R₇, R₈ and R₉ are all hydrogen or fluorine; R₁ and R₅ are independently selected from hydrogen, chlorine and fluorine with the proviso that R₁ and R₅ cannot be simultaneously hydrogen, R₃ selected from Cl, E is S, both R₂ and R₄ are hydrogen, and R₇, R₈ and R₉ are all hydrogen or fluorine; and R₁ and R₅ are independently selected from hydrogen, chlorine and fluorine with the proviso that R₁ and R₅ cannot be simultaneously hydrogen, R₃ is selected from Br, E is S, both R₂ and R₄ are hydrogen and R₇, R₈ and R₉ are all hydrogen or fluorine; R₁ and R₅ are independently selected from hydrogen and fluorine with the proviso that R₁ and R₅ cannot be simultaneously hydrogen, R₃ is selected from Cl, E is S, R₂ and R₄ both are hydrogen and R₇, R₈ and R₉ are hydrogen; R₁ and R₅ are independently selected from hydrogen, chlorine and fluorine with the proviso that R₁ and R₅ cannot be simultaneously hydrogen, R₃ is s Br, E is S, both R₂ and R₄ are hydrogen, and R₇, R₈ and R₉ are all hydrogen.

Also disclosed are compounds of Formula Ib or a salt thereof

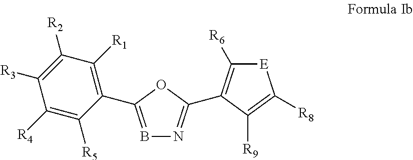

wherein,

R₁ and R₅ are independently selected from hydrogen, CH₃, F, Cl, Br, CF₃, and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and CO;

R₈ is selected from hydrogen and fluorine;

R₆ and R₉ are independently selected from hydrogen, F, Cl, CH₃, and OCF₃;

B is C(H) or C(CH₃); and

E is O or S.

In various embodiments of the compound of Formula Ib: R₁ and R₅ are independently selected from hydrogen, fluorine and chlorine with the proviso that R₁ and R₅ cannot be simultaneously hydrogen; R₁ and R₅ are independently selected from hydrogen, chlorine and fluorine with the proviso that R₁ and R₅ cannot be simultaneously hydrogen, and R₃ is selected from Cl, Br and F; R₁ and R₅ are independently selected from hydrogen, chlorine and fluorine with the proviso that R₁ and R₅ cannot be simultaneously hydrogen and R₃ is selected from Cl and Br; R₁ and R₅ are independently selected from hydrogen, chlorine and fluorine with the proviso that R₁ and R₅ cannot be simultaneously hydrogen, R₃ is selected from Cl, Br, F, and E is S; R₁ and R₅ are independently selected from hydrogen, chlorine and fluorine with the proviso that R₁ and R₅ cannot be simultaneously hydrogen, R₃ is selected from Cl, Br, and F, E is S, and both R₂ and R₄ are hydrogen; R₁ and R₅ are independently selected from hydrogen, chlorine and fluorine with the proviso that R₁ and R₅ cannot be simultaneously hydrogen, R₃ is selected from Cl, Br, F, E is S, both R₂ and R₄ are hydrogen and R₇, R₈ and R₉ are all hydrogen or fluorine; R₁ and R₅ are independently selected from hydrogen, chlorine and fluorine with the proviso that R₁ and R₅ cannot be simultaneously hydrogen, R₃ selected from Cl, E is S, both R₂ and R₄ are hydrogen, and R₇, R₈ and R₉ are all hydrogen or fluorine; and R₁ and R₅ are independently selected from hydrogen, chlorine and fluorine with the proviso that R₁ and R₅ cannot be simultaneously hydrogen, R₃ is selected from Br, E is S, both R₂ and R₄ are hydrogen and R₇, R₈ and R₉ are all hydrogen or fluorine; R₁ and R₅ are independently selected from hydrogen and fluorine with the proviso that R₁ and R₅ cannot be simultaneously hydrogen, R₃ is selected from Cl, E is S, R₂ and R₄ both are hydrogen and R₇, R₈ and R₉ are hydrogen; R₁ and R₅ are independently selected from hydrogen, chlorine and fluorine with the proviso that R₁ and R₅ cannot be simultaneously hydrogen, R₃ is s Br, E is S, both R₂ and R₄ are hydrogen, and R₇, R₈ and R₉ are all hydrogen.

Disclosed herein are compounds of Formula II or a salt thereof

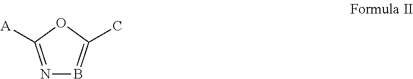

wherein,

A is selected from: phenyl, pyridyl, and pyrazyl, each of which can be optionally independently substituted with one or more substituents selected from: halogen, CF₃, CH₃, OCF₃, OCH₃, CN, and C(H)O;

B is C(H) or C(CH₃);

C is selected from: thienyl, furanyl, oxazolyl or isoxazolyl, each of which can be optionally independently substituted with one or more substituents selected from: fluorine, chlorine, CH₃, and OCF₃.

In various embodiments: A is phenyl; A is pyridyl; A is pyrazyl; B is C(H); B is C(CH₃); C is thienyl; C is furanyl; C is oxazolyl; and C is isoxazolyl.

Disclosed herein are compounds of Formula IIa

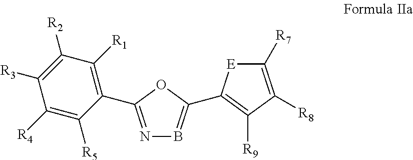

wherein,

R₁ and R₅ are independently selected from hydrogen, CH₃, F, Cl, Br, CF₃ and OCF₃

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and F;

R₉ is selected from hydrogen, F, Cl, CH₃, and OCF₃;

B is C(H) or C(CH₃); and

E is O or S.

In various embodiments of the compound of Formula IIa: R₁ and R₅ are independently selected from hydrogen, fluorine and chlorine with the proviso that R₁ and R₅ cannot be simultaneously hydrogen; R₁ and R₅ are independently selected from hydrogen, chlorine and fluorine with the proviso that R₁ and R₅ cannot be simultaneously hydrogen, and R₃ is selected from Cl, Br and F; R₁ and R₅ are independently selected from hydrogen, chlorine and fluorine with the proviso that R₁ and R₅ cannot be simultaneously hydrogen and R₃ is selected from Cl and Br; R₁ and R₅ are independently selected from hydrogen, chlorine and fluorine with the proviso that R₁ and R₅ cannot be simultaneously hydrogen, R₃ is selected from Cl, Br, F, and E is S; R₁ and R₅ are independently selected from hydrogen, chlorine and fluorine with the proviso that R₁ and R₅ cannot be simultaneously hydrogen, R₃ is selected from Cl, Br, and F, E is S, and both R₂ and R₄ are hydrogen; R₁ and R₅ are independently selected from hydrogen, chlorine and fluorine with the proviso that R₁ and R₅ cannot be simultaneously hydrogen, R₃ is selected from Cl, Br, F, E is S, both R₂ and R₄ are hydrogen and R₇, R₈ and R₉ are all hydrogen or fluorine; R₁ and R₅ are independently selected from hydrogen, chlorine and fluorine with the proviso that R₁ and R₅ cannot be simultaneously hydrogen, R₃ selected from Cl, E is S, both R₂ and R₄ are hydrogen, and R₇, R₈ and R₉ are all hydrogen or fluorine; and R₁ and R₅ are independently selected from hydrogen, chlorine and fluorine with the proviso that R₁ and R₅ cannot be simultaneously hydrogen, R₃ is selected from Br, E is S, both R₂ and R₄ are hydrogen and R₇, R₈ and R₉ are all hydrogen or fluorine; R₁ and R₅ are independently selected from hydrogen and fluorine with the proviso that R₁ and R₅ cannot be simultaneously hydrogen, R₃ is selected from Cl, E is S, R₂ and R₄ both are hydrogen and R₇, R₈ and R₉ are hydrogen; R₁ and R₅ are independently selected from hydrogen, chlorine and fluorine with the proviso that R₁ and R₅ cannot be simultaneously hydrogen, R₃ is s Br, E is S, both R₂ and R₄ are hydrogen, and R₇, R₈ and R₉ are all hydrogen.

Disclosed herein are compounds of Formula IIb or a salt thereof

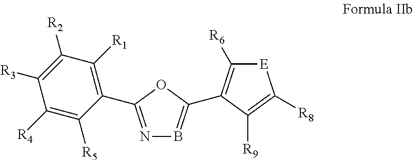

wherein,

R₁ and R₅ are independently selected from hydrogen, CH₃, F, Cl, Br, CF₃ and OCF₃;

R₂ and R₄ are independently selected from hydrogen, F, Cl, Br and CF₃;

R₃ is selected from hydrogen, CH₃, CF₃, F, Cl, Br, OCF₃, OCH₃, CN and C(H)O;

R₈ is selected from hydrogen and fluorine;

R₆ and R₉ are independently selected from hydrogen, F, Cl, CH₃ and OCF₃;

B is C(H) or C(CH₃); and

E is O or S.

In various embodiments of the compound of Formula IIb: R₁ and R₅ are independently selected from hydrogen, fluorine and chlorine with the proviso that R₁ and R₅ cannot be simultaneously hydrogen; R₁ and R₅ are independently selected from hydrogen, chlorine and fluorine with the proviso that R₁ and R₅ cannot be simultaneously hydrogen, and R₃ is selected from Cl, Br and F; R₁ and R₅ are independently selected from hydrogen, chlorine and fluorine with the proviso that R₁ and R₅ cannot be simultaneously hydrogen and R₃ is selected from Cl and Br; R₁ and R₅ are independently selected from hydrogen, chlorine and fluorine with the proviso that R₁ and R₅ cannot be simultaneously hydrogen, R₃ is selected from Cl, Br, F, and E is S; R₁ and R₅ are independently selected from hydrogen, chlorine and fluorine with the proviso that R₁ and R₅ cannot be simultaneously hydrogen, R₃ is selected from Cl, Br, and F, E is S, and both R₂ and R₄ are hydrogen; R₁ and R₅ are independently selected from hydrogen, chlorine and fluorine with the proviso that R₁ and R₅ cannot be simultaneously hydrogen, R₃ is selected from Cl, Br, F, E is S, both R₂ and R₄ are hydrogen and R₇, R₈ and R₉ are all hydrogen or fluorine; R₁ and R₅ are independently selected from hydrogen, chlorine and fluorine with the proviso that R₁ and R₅ cannot be simultaneously hydrogen, R₃ selected from Cl, E is S, both R₂ and R₄ are hydrogen, and R₇, R₈ and R₉ are all hydrogen or fluorine; and R₁ and R₅ are independently selected from hydrogen, chlorine and fluorine with the proviso that R₁ and R₅ cannot be simultaneously hydrogen, R₃ is selected from Br, E is S, both R₂ and R₄ are hydrogen and R₇, R₈ and R₉ are all hydrogen or fluorine; R₁ and R₅ are independently selected from hydrogen and fluorine with the proviso that R₁ and R₅ cannot be simultaneously hydrogen, R₃ is selected from Cl, E is S, R₂ and R₄ both are hydrogen and R₇, R₈ and R₉ are hydrogen; R₁ and R₅ are independently selected from hydrogen, chlorine and fluorine with the proviso that R₁ and R₅ cannot be simultaneously hydrogen, R₃ is s Br, E is S, both R₂ and R₄ are hydrogen, and R₇, R₈ and R₉ are all hydrogen.

Disclosed herein are compounds of Formula III or a salt thereof

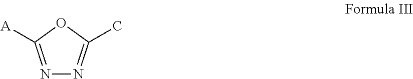

wherein,

A is phenyl, pyridyl, or pyrazyl, each of which can be optionally independently substituted with one or more substituents selected from: halogen, CF₃, CH₃, OCF₃, OCH₃, CN and C(H)O.

C is thienyl, furanyl, oxazolyl or isoxazolyl each of which can be optionally independently substituted with one or more substituents selected from: fluorine, chlorine, CH₃ and OCF₃.

In various embodiments: A is phenyl; A is pyridyl; A is pyrazyl; C is thienyl; C is furanyl; C is oxazolyl; and C is isoxazolyl.

Also disclosed are compounds of Formula Ma or a salt thereof

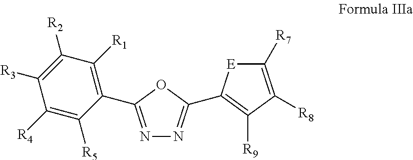

wherein,

R₁ and R₅ are independently selected from hydrogen, CH₃, F, Cl, Br, CF₃, and OCF₃,

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃; and

E is O or S.

In various embodiments of the compound of Formula Ma: R₁ and R₅ are independently selected from hydrogen and CH₃ with the proviso that R₁ and R₅ cannot be simultaneously hydrogen, R₃ fluorine, chlorine or bromine, and E is O; R₁ and R₅ are independently selected from hydrogen and CH₃ with the proviso that R₁ and R₅ cannot be simultaneously hydrogen, R₃ fluorine, chlorine and bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, and E is O; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is S, and R₉ is hydrogen or fluorine; R₁ and R₅ are independently selected from hydrogen and Cl, R₃ is fluorine, chlorine or bromine, E is O, and R₉ is fluorine; R₁ and R₅ are independently selected from hydrogen and CH₃ with the proviso that R₁ and R₅ cannot be simultaneously hydrogen, R₃ is fluorine, chlorine or bromine, and E is O; R₁ and R₅ are independently selected from hydrogen and CH₃ with the proviso that R₁ and R₅ cannot be simultaneously hydrogen, R₃ fluorine, chlorine or bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, and E is O; R₁ and R₅ are independently selected from hydrogen and CH₃ with the proviso that R₁ and R₅ cannot be simultaneously hydrogen, R₂ and R₄ are hydrogen, R₃ chlorine or bromine, E is S and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, and E is O and R₇, R₈ and R₉ are hydrogen; and R₁ and R₅ are independently selected from hydrogen and Cl, R₃ fluorine, chlorine and bromine, E is O and R₉ is fluorine.

Also disclosed are compounds of Formula IIIb or a salt thereof

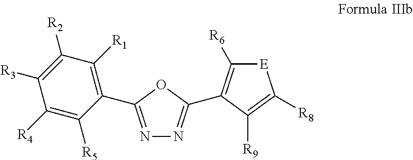

wherein,

R₁ and R₅ are independently selected from hydrogen, CH₃, F, Cl, Br, CF₃, and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₈ is selected from hydrogen and fluorine;

R₆ and R₉ are independently selected from hydrogen, F, Cl, CH₃, and OCF₃; and

E is O or S.

In various embodiments of the compound of Formula IIIb: R₁ and R₅ are independently selected from hydrogen and CH₃ with the proviso that R₁ and R₅ cannot be simultaneously hydrogen, R₃ fluorine, chlorine or bromine, and E is O; R₁ and R₅ are independently selected from hydrogen and CH₃ with the proviso that R₁ and R₅ cannot be simultaneously hydrogen, R₃ fluorine, chlorine and bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, and E is O; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is S, and R₉ is hydrogen or fluorine; R₁ and R₅ are independently selected from hydrogen and Cl, R₃ is fluorine, chlorine or bromine, E is O, and R₉ is fluorine; R₁ and R₅ are independently selected from hydrogen and CH₃ with the proviso that R₁ and R₅ cannot be simultaneously hydrogen, R₃ is fluorine, chlorine or bromine, and E is O; R₁ and R₅ are independently selected from hydrogen and CH₃ with the proviso that R₁ and R₅ cannot be simultaneously hydrogen, R₃ fluorine, chlorine or bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, and E is O; R₁ and R₅ are independently selected from hydrogen and CH₃ with the proviso that R₁ and R₅ cannot be simultaneously hydrogen, R₂ and R₄ are hydrogen, R₃ chlorine or bromine, E is S and R₉ is hydrogen or fluorine; and R₁ and R₅ are independently selected from hydrogen and Cl, R₃ is fluorine, chlorine or bromine, E is O and R₉ is fluorine.

Also disclosed are compounds of Formula (IV) or a salt thereof

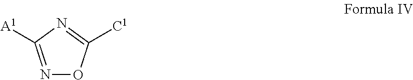

wherein,

A^{l} is phenyl, pyridyl, pyrazyl, oxazolyl or isoxazolyl each of which can be optionally independently substituted with one or more substituents selected from: halogen, CF₃, CH₃, OCF₃, OCH₃, CN, and C(H)O; and

C^{l} is thienyl, furanyl, oxazolyl or isoxazolyl, each of which can be optionally independently substituted with one or more substituents selected from fluorine, chlorine, CH₃, and OCF₃

In various embodiments: A^{l} is phenyl; A^{l} is pyridyl; A^{l} is pyrazyl; A^{l} is oxazolyl; A^{l} is isoxazolyl; C^{l} is thienyl; C^{l} is furanyl; C^{l} is oxazolyl; and C^{l} is isoxazolyl.

Also disclosed are compounds of Formula IVa or a salt thereof

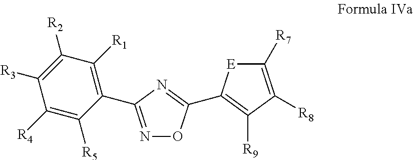

wherein,

R₁ and R₅ are independently selected from hydrogen, CH₃, F, Cl, Br, CF₃ and OCF₃,

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃; and

E is O or S.

In various embodiments of the compound of Formula IVa: R₁ and R₅ are independently selected from hydrogen and CH₃ with the proviso that R₁ and R₅ cannot be simultaneously hydrogen, R₃ is fluorine, chlorine or bromine, and E is O; R1 and R5 are independently selected from hydrogen and CH3 with the proviso that R1 and R5 cannot be simultaneously hydrogen, both R2 and R4 are hydrogen, R3 is chlorine or bromine, and E is O and R7, R8 and R9 are hydrogen; R₁ and R₅ are independently selected from hydrogen and CH₃ with the proviso that R₁ and R₅ cannot be simultaneously hydrogen, R₃ is fluorine, chlorine or bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is O; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is S, and R₉ is hydrogen or fluorine; R₁ and R₅ are independently selected from hydrogen and Cl, R₃ is fluorine, chlorine or bromine, E is O, and R₉ is fluorine.

Also disclosed are compounds of Formula IVb or a salt thereof

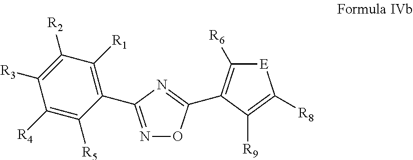

wherein,

R₁ and R₅ are independently selected from hydrogen, CH₃, F, Cl, Br, CF₃ and OCF₃,

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₈ is selected from hydrogen and fluorine;

R₆ and R₉ are independently selected from hydrogen, F, Cl, CH₃, and OCF₃; and

E is O or S.

In various embodiments of the compound of Formula IVb: R₁ and R₅ are independently selected from hydrogen and CH₃ with the proviso that R₁ and R₅ cannot be simultaneously hydrogen, R₃ is fluorine, chlorine or bromine, and E is O; R₁ and R₅ are independently selected from hydrogen and CH₃ with the proviso that R₁ and R₅ cannot be simultaneously hydrogen, R₃ is fluorine, chlorine or bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is O; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is S, and R₉ is hydrogen or fluorine; and R₁ and R₅ are independently selected from hydrogen and Cl, R₃ is fluorine, chlorine or bromine, E is O, and R₉ is fluorine.

Disclosed herein are compounds of Formula (V) or a salt thereof

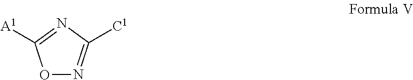

wherein,

A^{l} is phenyl, pyridyl, pyrazyl, oxazolyl or isoxazolyl, each of which can be optionally independently substituted with one or more substituents selected from: halogen, CF₃, CH₃, OCF₃, OCH₃, CN, and C(H)O; and

C^{l} is thienyl, furanyl, oxazolyl or isoxazolyl, each of which can be optionally independently substituted with one or more substituents selected from fluorine, chlorine, CH₃, and OCF₃.

In various embodiments: A^{l} is phenyl; A^{l} is pyridyl; A^{l} is pyrazyl; A^{l} is oxazolyl; A^{l} is isoxazolyl; C^{l} is thienyl; C^{l} is furanyl; C^{l} is oxazolyl; and C^{l} is isoxazolyl.

Also disclosed are compounds of Formula Va or a salt thereof

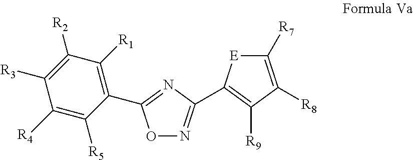

wherein,

R₁ and R₅ are independently selected from hydrogen, CH₃, F, Cl, Br, CF₃ and OCF₃,

with the proviso that R₁ and R₅ cannot be simultaneously hydrogen;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃; and

E is O or S.

In various embodiments of the compound of Formula Va: R₁ and R₅ are independently selected from hydrogen and CH₃ with the proviso that R₁ and R₅ cannot be simultaneously hydrogen, R₃ is fluorine, chlorine or bromine, E is S, and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is O; R₁ and R₅ are independently selected from hydrogen and CH₃ with the proviso that R₁ and R₅ cannot be simultaneously hydrogen, both R₂ and R₄ are hydrogen, R₃ chlorine or bromine, E is S, and R₉ is hydrogen or fluorine; R₁ and R₅ are independently selected from hydrogen and Cl, R₃ is fluorine, chlorine or bromine, E is O, and R₉ is fluorine.

Also disclosed are compounds of Formula Vb or a salt thereof

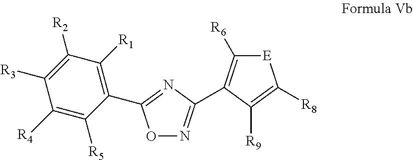

wherein,

R₁ and R₅ are independently selected from hydrogen, CH₃, F, Cl, Br, CF₃ and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₈ is selected from hydrogen and fluorine;

R₆ and R₉ are independently selected from hydrogen, F, Cl, CH₃, and OCF₃; and

E is O or S.

In various embodiments of the compound of Formula Vb: R₁ and R₅ are independently selected from hydrogen and CH₃ with the proviso that R₁ and R₅ cannot be simultaneously hydrogen, R₃ is fluorine, chlorine and bromine, and E is O; R₁ and R₅ are independently selected from hydrogen and CH₃ with the proviso that R₁ and R₅ cannot be simultaneously hydrogen, R₃ is fluorine, chlorine or bromine, E is S and R₉ is hydrogen or fluorine; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is O; R₁ and R₅ are independently selected from hydrogen and CH₃ with the proviso that R₁ and R₅ cannot be simultaneously hydrogen, both R₂ and R₄ are hydrogen, R₃ is chlorine or bromine, E is S, and R₉ is hydrogen or fluorine; R₁ and R₅ are independently selected from hydrogen and Cl, R₃ is fluorine, chlorine or bromine, E is O, and R₉ is fluorine.

Described herein are compounds of Formula (VI) or a salt thereof

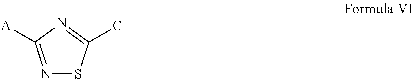

wherein,

A is an optionally substituted aryl or optionally independently singly or multiply substituted arylalkyl or optionally independently singly or multiply substituted heteroaryl or optionally independently singly or multiply substituted heteroarylalkyl wherein the substituents are selected from the group consisting of halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆) alkynyl, C₁-C₆ hydroxyalkyl, amino, ureido, cyano, C₁-C₆ acylamino, hydroxy, thiol, C₁-C₆ acyloxy, azido, C₁-C₆ alkoxy and carboxy, and C(H)O;

C is a thienyl, furanyl, oxazolyl or isoxazolyl each of which can be optionally independently substituted with one or more with substituents selected from: fluorine, chlorine, CH₃, and OCF₃.

In various embodiments: A is aryl; A is arylalkyl; A is heteroarylalkyl; A is heteroaryl; C is thienyl; C is furanyl; C is oxazolyl; C is isoxazolyll; A is pyridyl; A is pyrazyl; A is oxazolyl; and A is isoxazolyl; compounds in which both A and C are not thiophenyl and compounds in which both A and C are not furanyl.

Also described herein are compounds of Formula VIa or a salt thereof

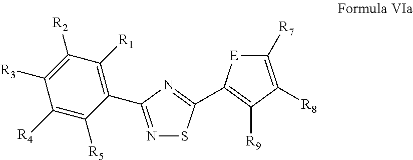

wherein,

R₁ and R₅ are independently selected from hydrogen, CH₃, F, Cl, Br, CF₃ and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃; and

E is O or S.

In various embodiments of the compound of Formula VIa: R₁ and R₅ are independently selected from hydrogen, CH₃, F and Cl; R₁ and R₅ are independently selected from hydrogen, CH₃, fluorine and Cl, and R₃ is Cl; R₁ and R₅ are independently selected from hydrogen, CH₃, fluorine and Cl, and R₃ is Br, R₁ and R₅ are independently selected from hydrogen, CH₃, fluorine and Cl, R₃ is Cl, and E is O; R₁ and R₅ are independently selected from hydrogen, CH₃, fluorine and Cl, and R₃ is Br and E is O; R₁ and R₅ are independently selected from hydrogen and CH₃, and R₃ is Cl, E is O, and R₆, R₈ and R₉ are selected from hydrogen and fluorine; R₁ and R₅ are independently selected from hydrogen and CH₃, and R₃ is Br, E is O, and R₇, R₈ and R₉ are selected from hydrogen and fluorine; R₁ and R₅ are independently selected from hydrogen and fluorine, and R₃ is Cl, E is O, and R₇, R₈ and R₉ are selected from hydrogen and fluorine; R₁ and R₅ are independently selected from hydrogen and fluorine, R₃ is Br, E is O, and R₇, R₈ and R₉ are selected from hydrogen and fluorine; R₁ and R₅ are independently selected from hydrogen and chlorine, and R₃ is Cl, E is O, and R₇, R₈ and R₉ are selected from hydrogen and fluorine; R₁ and R₅ are independently selected from hydrogen and CH₃, R₃ is Cl, R₂ and R₄ are hydrogen E is O, and R₇, R₈ and R₉ are selected from hydrogen and fluorine; R₁ and R₅ are independently selected from hydrogen and CH₃, R₂ and R₄ are hydrogen, R₃ is Br, E is O, and R₇, R₈ and R₉ are selected from hydrogen and fluorine; R₁ and R₅ are independently selected from hydrogen and fluorine, R₂ and R₄ are hydrogen, R₃ is Cl, E is O, and R₇, R₈ and R₉ are selected from hydrogen and fluorine; R₁ and R₅ are independently selected from hydrogen and fluorine, R₂ and R₄ are hydrogen, R₃ is Br, E is O, and R₇, R₈ and R₉ are selected from hydrogen and fluorine; R₁ and R₅ are independently selected from hydrogen and chlorine, R₂ and R₄ are hydrogen, R₃ is Cl, E is O, and R₇, R₈ and R₉ are selected from hydrogen and fluorine.

Also described herein are compounds of Formula VIb or a salt thereof

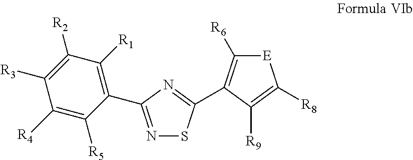

wherein,

R₁ and R₅ are independently selected from hydrogen, CH₃, F, Cl, Br, CF₃, and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₈ is selected from hydrogen and fluorine;

R₆ and R₉ are independently selected from hydrogen, F, Cl, CH₃, and OCF₃; and

E is O or S.

In various embodiments of the compound of Formula VIb: R₁ and R₅ are independently selected from hydrogen, CH₃, F and Cl; R₁ and R₅ are independently selected from hydrogen, CH₃, fluorine and Cl, and R₃ is Cl; R₁ and R₅ are independently selected from hydrogen, CH₃, fluorine and Cl, and R₃ is Br; R₁ and R₅ are independently selected from hydrogen, CH₃, fluorine and Cl, R₃ is Cl, and E is O; R₁ and R₅ are independently selected from hydrogen, CH₃, fluorine and Cl, and R₃ is Br and E is O; R₁ and R₅ are independently selected from hydrogen and CH₃, and R₃ is Cl, E is O, and R₆, R₈ and R₉ are selected from hydrogen and fluorine; R₁ and R₅ are independently selected from hydrogen and CH₃, and R₃ is Br, E is O, and R₇, R₈ and R₉ are selected from hydrogen and fluorine; R₁ and R₅ are independently selected from hydrogen and fluorine, and R₃ is Cl, E is O, and R₇, R₈ and R₉ are selected from hydrogen and fluorine; R₁ and R₅ are independently selected from hydrogen and fluorine, R₃ is Br, E is O, and R₇, R₈ and R₉ are selected from hydrogen and fluorine; R₁ and R₅ are independently selected from hydrogen and chlorine, and R₃ is Cl, E is O, and R₇, R₈ and R₉ are selected from hydrogen and fluorine; R₁ and R₅ are independently selected from hydrogen and CH₃, R₃ is Cl, R₂ and R₄ are hydrogen E is O, and R₇, R₈ and R₉ are selected from hydrogen and fluorine; R₁ and R₅ are independently selected from hydrogen and CH₃, R₂ and R₄ are hydrogen, R₃ is Br, E is O, and R₇, R₈ and R₉ are selected from hydrogen and fluorine; R₁ and R₅ are independently selected from hydrogen and fluorine, R₂ and R₄ are hydrogen, R₃ is Cl, E is O, and R₇, R₈ and R₉ are selected from hydrogen and fluorine; R₁ and R₅ are independently selected from hydrogen and fluorine, R₂ and R₄ are hydrogen, R₃ is Br, E is O, and R₇, R₈ and R₉ are selected from hydrogen and fluorine; R₁ and R₅ are independently selected from hydrogen and chlorine, R₂ and R₄ are hydrogen, R₃ is Cl, E is O, and R₇, R₈ and R₉ are selected from hydrogen and fluorine.

Described herein are compounds of Formula (VII) or a salt thereof

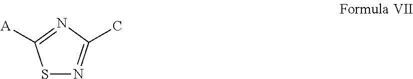

wherein,

A is an optionally substituted aryl or optionally independently singly or multiply substituted arylalkyl (e.g., arylC₁₋₃alkyl or arylC₁-C₆) or optionally independently singly or multiply substituted heteroaryl or optionally independently singly or multiply substituted heteroarylalkyl (e.g., heteroaryl C₁₋₃alkyl or heteroaryl C₁-C₆) wherein the substituents are selected from the group consisting of halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆) alkynyl, C₁-C₆ hydroxyalkyl, amino, ureido, cyano, C₁-C₆ acylamino, hydroxy, thiol, C₁-C₆ acyloxy, azido, C₁-C₆ alkoxy and carboxy, C(H)O;

C is a thienyl, furanyl, oxazolyl or isoxazolyl each of which can be optionally independently substituted with one or more with substituents selected from: fluorine, chlorine, CH₃, and OCF₃.

In various embodiments: A is aryl; A is arylalkyl; A is heteroarylalkyl; A is heteroaryl; C is thienyl; C is furanyl; C is oxazolyl; C is isoxazolyl; A is pyridyl; A is pyrazyl; A is oxazolyl; and A is isoxazolyl; both A and C are not thiophenyl; and both A and C are not furanyl.

Also described herein is a compound having Formula VIIa or a salt thereof

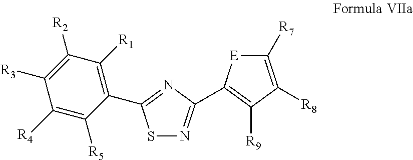

wherein,

R₁ and R₅ are independently selected from hydrogen, CH₃, F, Cl, Br, CF₃, and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₇ and R₈ are independently selected from hydrogen and fluorine;

R₉ is selected from hydrogen, F, Cl, CH₃, and OCF₃;

E is O or S.

In various embodiments of the compound of Formula VIIa: R₁ and R₅ are independently selected from hydrogen, CH₃, F and Cl; R₁ and R₅ are independently selected from hydrogen, CH₃, fluorine and Cl, and R₃ is Cl; R₁ and R₅ are independently selected from hydrogen, CH₃, fluorine and Cl, and R₃ is Br, R₁ and R₅ are independently selected from hydrogen, CH₃, fluorine and Cl, R₃ is Cl, and E is O; R₁ and R₅ are independently selected from hydrogen, CH₃, fluorine and Cl, and R₃ is Br and E is O; R₁ and R₅ are independently selected from hydrogen and CH₃, and R₃ is Cl, E is O, and R₆, R₈ and R₉ are selected from hydrogen and fluorine; R₁ and R₅ are independently selected from hydrogen and CH₃, and R₃ is Br, E is O, and R₇, R₈ and R₉ are selected from hydrogen and fluorine; R₁ and R₅ are independently selected from hydrogen and fluorine, and R₃ is Cl, E is O, and R₇, R₈ and R₉ are selected from hydrogen and fluorine; R₁ and R₅ are independently selected from hydrogen and fluorine, R₃ is Br, E is O, and R₇, R₈ and R₉ are selected from hydrogen and fluorine; R₁ and R₅ are independently selected from hydrogen and chlorine, and R₃ is Cl, E is O, and R₇, R₈ and R₉ are selected from hydrogen and fluorine; R₁ and R₅ are independently selected from hydrogen and CH₃, R₃ is Cl, R₂ and R₄ are hydrogen E is O, and R₇, R₈ and R₉ are selected from hydrogen and fluorine; R₁ and R₅ are independently selected from hydrogen and CH₃, R₂ and R₄ are hydrogen, R₃ is Br, E is O, and R₇, R₈ and R₉ are selected from hydrogen and fluorine; R₁ and R₅ are independently selected from hydrogen and fluorine, R₂ and R₄ are hydrogen, R₃ is Cl, E is O, and R₇, R₈ and R₉ are selected from hydrogen and fluorine; R₁ and R₅ are independently selected from hydrogen and fluorine, R₂ and R₄ are hydrogen, R₃ is Br, E is O, and R₇, R₈ and R₉ are selected from hydrogen and fluorine; R₁ and R₅ are independently selected from hydrogen and chlorine, R₂ and R₄ are hydrogen, R₃ is Cl, E is O, and R₇, R₈ and R₉ are selected from hydrogen and fluorine.

Also described herein is a compound having Formula VIIb or a salt thereof

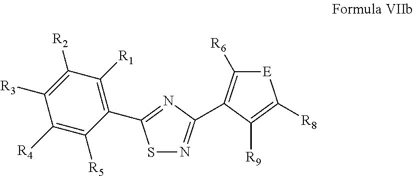

wherein,

R₁ and R₅ are independently selected from hydrogen, CH₃, F, Cl, Br, CF₃ and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and C(H)O;

R₈ is selected from hydrogen and fluorine;

R₆ and R₉ are independently selected from hydrogen, F, Cl, CH₃, and OCF₃; and

E is O or S.

In various embodiments of the compound of Formula VIIa: R₁ and R₅ are independently selected from hydrogen, CH₃, F and Cl; R₁ and R₅ are independently selected from hydrogen, CH₃, fluorine and Cl, and R₃ is Cl; R₁ and R₅ are independently selected from hydrogen, CH₃, fluorine and Cl, and R₃ is Br; R₁ and R₅ are independently selected from hydrogen, CH₃, fluorine and Cl, R₃ is Cl, and E is O; R₁ and R₅ are independently selected from hydrogen, CH₃, fluorine and Cl, and R₃ is Br and E is O; R₁ and R₅ are independently selected from hydrogen and CH₃, and R₃ is Cl, E is O, and R₆, R₈ and R₉ are selected from hydrogen and fluorine; R₁ and R₅ are independently selected from hydrogen and CH₃, and R₃ is Br, E is O, and R₇, R₈ and R₉ are selected from hydrogen and fluorine; R₁ and R₅ are independently selected from hydrogen and fluorine, and R₃ is Cl, E is O, and R₇, R₈ and R₉ are selected from hydrogen and fluorine; R₁ and R₅ are independently selected from hydrogen and fluorine, R₃ is Br, E is O, and R₇, R₈ and R₉ are selected from hydrogen and fluorine; R₁ and R₅ are independently selected from hydrogen and chlorine, and R₃ is Cl, E is O, and R₇, R₈ and R₉ are selected from hydrogen and fluorine; R₁ and R₅ are independently selected from hydrogen and CH₃, R₃ is Cl, R2 and R4 are hydrogen E is O, and R₇, R₈ and R₉ are selected from hydrogen and fluorine; R₁ and R₅ are independently selected from hydrogen and CH₃, R₂ and R₄ are hydrogen, R₃ is Br, E is O, and R₇, R₈ and R₉ are selected from hydrogen and fluorine; R₁ and R₅ are independently selected from hydrogen and fluorine, R₂ and R₄ are hydrogen, R₃ is Cl, E is O, and R₇, R₈ and R₉ are selected from hydrogen and fluorine; R₁ and R₅ are independently selected from hydrogen and fluorine, R₂ and R₄ are hydrogen, R₃ is Br, E is O, and R₇, R₈ and R₉ are selected from hydrogen and fluorine; R₁ and R₅ are independently selected from hydrogen and chlorine, R₂ and R₄ are hydrogen, R₃ is Cl, E is O, and R₇, R₈ and R₉ are selected from hydrogen and fluorine.

Also described herein is a method for control of soybean sudden death syndrome, the method comprising administering to a plant, a seed, or soil a composition comprising an effective amount of a compound of any of Formulas I, Ia, Ib, II, IIa, IIb, III, IIIa, IIIb, IV, IVa, IVb, V, Va, Vb, VI, VIa, VIb, VII, VIIa and VIIb without the provisos.

Also described herein is a method for control of soybean sudden death syndrome, the method comprising administering to a plant, a seed, or soil a composition comprising an effective amount of a compound of any of Formulas I, Ia, Ib, II, IIa, IIb, III, IIIa, IIIb, IV, IVa, IVb, V, Va, Vb, VI, VIa, VIb, VII, VIIa and VIIb with the provisos.

Also described is a composition comprising a compound of any of Formulas I, Ia, Ib, II, IIa, IIb, III, IIIa, IIIb, IV, IVa, IVb, V, Va, Vb, VI, VIa, VIb, VII, VIIa and VIIb without the provisos, at a concentration sufficient to reduce the severity of soybean sudden death syndrome when applied to a plant, a seed, or soil.

Also described is a composition comprising a compound of any of Formulas I, Ia, Ib, II, IIa, IIb, III, IIIa, IIIb, IV, IVa, IVb, V, Va, Vb, VI, VIa, VIb, VII, VIIa and VIIb with the provisos, at a concentration sufficient to reduce the severity of soybean sudden death syndrome when applied to a plant, a seed, or soil.

Also described is a composition comprising: oxazole, oxadiazole or thiadiazole analogs or mixtures of analogs selected from the group consisting of the compounds 3-phenyl-5-(thiophen-2-yl)-1,2,4-oxadiazole, 3-(4-fluorophenyl)-5-(thiophen-2-yl)-1,2,4-oxadiazole, 3-(4-chlorophenyl)-5-(furan-2-yl)-1,2,4-oxadiazole, 3-(4-chlorophenyl)-5-(thiophen-2-yl)-1,2,4-oxadiazole, 3-(4-chloro-2-methylphenyl)-5-(furan-2-yl)-1,2,4-oxadiazole, 5-(4-chloro-2-methylphenyl)-3-(furan-2-yl)-1,2,4-oxadiazole, 3-(4-bromo-2-methylphenyl)-5-(furan-2-yl)-1,2,4-oxadiazole, 3-(4-fluoro-2-methylphenyl)-5-(thiophen-2-yl)-1,2,4-oxadiazole, difluorophenyl)-5-(thiophen-2-yl)-1,2,4-oxadiazole, 3-(4-bromo-2-fluorophenyl)-5-(thiophen-2-yl)-1,2,4-oxadiazole, 5-(thiophen-2-yl)-3-(2,4,6-trifluorophenyl)-1,2,4-oxadiazole, 3-(2,4-dichlorophenyl)-5-(furan-2-yl)-1,2,4-oxadiazole, 3-(4-bromo-2-chlorophenyl)-5-(furan-2-yl)-1,2,4-oxadiazole, 3-(2-chloro-4-fluorophenyl)-5-(thiophen-2-yl)-1,2,4-oxadiazole, 3-(4-chlorophenyl)-5-(thiophen-2-yl)-1,2,4-thiadiazole, 3-(4-chlorophenyl)-5-(furan-2-yl)-1,2,4-thiadiazole, 3-(4-chlorophenyl)-5-(3-methylfuran-2-yl)-1,2,4-oxadiazole, 5-(4-chloro-2-fluorophenyl)-2-(thiophen-2-yl)oxazole, 2-(4-chloro-2-fluorophenyl)-5-(thiophen-2-yl)oxazole, 5-(4-chloro-2-fluorophenyl)-2-(furan-2-yl)oxazole, 5-(4-chloro-2-methylphenyl)-2-(furan-3-yl)oxazole, 3-(4-chloro-2-methylphenyl)-5-(furan-2-yl)-1,2,4-thiadiazole, 5-(4-chloro-2-methylphenyl)-3-(furan-2-yl)-1,2,4-thiadiazole, 3-(4-bromo-2-methylphenyl)-5-(furan-2-yl)-1,2,4-thiadiazole, 5-(furan-2-yl)-3-(4-methoxy-2-methylphenyl)-1,2,4-oxadiazole, 3-(6-chloropyridin-3-yl)-5-(thiophen-2-yl)-1,2,4-thiadiazole, 3-(6-chloropyridin-3-yl)-5-(furan-2-yl)-1,2,4-thiadiazole, 5-(2,4-difluorophenyl)-2-(thiophen-2-yl)oxazole, 5-(2,4-difluorophenyl)-2-(furan-2-yl)oxazole, 5-(4-bromo-2-fluorophenyl)-2-(thiophen-2-yl)oxazole, 5-(4-bromo-2-fluorophenyl)-2-(furan-2-yl)oxazole, 3-(2,4-difluorophenyl)-5-(furan-2-yl)-1,2,4-thiadiazole, 3-(4-chloro-2-fluorophenyl)-5-(furan-2-yl)-1,2,4-thiadiazole, 3-(4-bromo-2-fluorophenyl)-5-(furan-2-yl)-1,2,4-thiadiazole, 3-(2,4-difluorophenyl)-5-(thiophen-2-yl)-1,2,4-thiadiazole, 3-(4-chloro-2-fluorophenyl)-5-(thiophen-2-yl)-1,2,4-thiadiazole, 3-(4-bromo-2-fluorophenyl)-5-(thiophen-2-yl)-1,2,4-thiadiazole, 5-(furan-2-yl)-3-(4-methoxy-2-methylphenyl)-1,2,4-thiadiazole, 3-(2,4-dichlorophenyl)-5-(furan-2-yl)-1,2,4-thiadiazole, 3-(4-bromo-2-chlorophenyl)-5-(furan-2-yl)-1,2,4-thiadiazole, 3-(2,6-dichloropyridin-3-yl)-5-(furan-2-yl)-1,2,4-thiadiazole, 5-(2,4-dichlorophenyl)-2-(thiophen-2-yl)oxazole, 3-(4-chlorophenyl)-5-(thiophen-3-yl)-1,2,4-oxadiazole, 5-(4-chloro-2-methylphenyl)-2-(furan-3-yl)oxazole.

Adjuvants and Excipients

Generally, the compositions described herein can comprise any adjuvants, excipients, or other desirable components known in the art.

For example, in some embodiments, the composition further comprises a surfactant.

Examples of anionic surfactants include alkyl sulfates, alcohol sulfates, alcohol ether sulfates, alpha olefin sulfonates, alkylaryl ether sulfates, arylsulfonates, alkylsulfonates, alkylaryl sulfonates, sulfosuccinates, mono- or diphosphate esters of polyalkoxylated alkyl alcohols or alkyl phenols, mono- or disulfosuccinate esters of alcohols or polyalkoxylated alkanols, alcohol ether carboxylates, phenol ether carboxylates. In one embodiment, the surfactant is an alkylaryl sulfonate.

Non-limiting examples of commercially available anionic surfactants include sodium dodecylsulfate (Na-DS, SDS), MORWET D-425 (a sodium salt of alkyl naphthalene sulfonate condensate, available from Akzo Nobel), MORWET D-500 (a sodium salt of alkyl naphthalene sulfonate condensate with a block copolymer, available from Akzo Nobel), sodium dodecylbenzene sulfonic acid (Na-DBSA) (available from Aldrich), diphenyloxide disulfonate, naphthalene formaldehyde condensate, DOWFAX (available from Dow), dihexylsulfosuccinate, and dioctylsulfosuccinate, alkyl naphthalene sulfonate condensates, and salts thereof

Examples of non-ionic surfactants include sorbitan esters, ethoxylated sorbitan esters, alkoxylated alkylphenols, alkoxylated alcohols, block copolymer ethers, and lanolin derivatives. In accordance with one embodiment, the surfactant comprises an alkylether block copolymer.

Non-limiting examples of commercially available non-ionic surfactants include SPAN 20, SPAN 40, SPAN 80, SPAN 65, and SPAN 85 (available from Aldrich); TWEEN 20, TWEEN 40, TWEEN 60, TWEEN 80, and TWEEN 85 (available from Aldrich); IGEPAL CA-210, IGEPAL CA-520, IGEPAL CA-720, IGEPAL CO-210, IGEPAL CO-520, IGEPAL CO-630, IGEPAL CO-720, IGEPAL CO-890, and IGEPAL DM-970 (available from Aldrich); Triton X-100 (available from Aldrich); BRIJ S10, BRIJ S20, BRIJ 30, BRIJ 52, BRIJ 56, BRIJ 58, BRIJ 72, BRIJ 76, BRIJ 78, BRIJ 92V, BRIJ 97, and BRIJ 98 (available from Aldrich); PLURONIC L-31, PLURONIC L-35, PLURONIC L-61, PLURONIC L-81, PLURONIC L-64, PLURONIC L-121, PLURONIC 10R5, PLURONIC 17R4, and PLURONIC 31R1 (available from Aldrich); Atlas G-5000 and Atlas G-5002L (available from Croda); ATLOX 4912 and ATLOX 4912-SF (available from Croda); and SOLUPLUS (available from BASF), LANEXOL AWS (available from Croda).

Non-limiting examples of cationic surfactants include mono alkyl quaternary amine, fatty acid amide surfactants, amidoamine, imidazoline, and polymeric cationic surfactants.

In some embodiments, the composition comprises a co-solvent in addition to water. Non-limiting examples of co-solvents that can be used include ethyl lactate, methyl soyate/ethyl lactate co-solvent blends (e.g., STEPOSOL, available from Stepan), isopropanol, acetone, 1,2-propanediol, n-alkylpyrrolidones (e.g., the AGSOLEX series, available from ISP), a petroleum based-oil (e.g., AROMATIC series and SOLVESSO series available from Exxon Mobil), isoparaffinic fluids (e.g. ISOPAR series, available from Exxon Mobil), cycloparaffinic fluids (e.g. NAPPAR 6, available from Exxon Mobil), mineral spirits (e.g. VARSOL series available from Exxon Mobil), and mineral oils (e.g., paraffin oil).

Examples of commercially available organic solvents include pentadecane, ISOPAR M, ISOPAR V, and ISOPAR L (available from Exxon Mobil).

The composition can be produced in concentrated form that includes little or no water. The composition can be diluted with water or some other solvent prior to use.

Additional Active Ingredients

In some embodiments, the compositions described herein may comprise one or more additional active ingredients.

In addition to the compounds (e.g., oxazoles, oxadiazoles, and thiadiazoles) described herein, compositions and formulations in some embodiments may further comprise one or more pesticidal agents. Pesticidal agents include chemical pesticides and biopesticides or biocontrol agents. Various types of chemical pesticides and biopesticides include acaricides, insecticides, nematicides, fungicides, gastropodicides, herbicides, virucides, bactericides, and combinations thereof. Biopesticides or biocontrol agents may include bacteria, fungi, beneficial nematodes, and viruses that exhibit pesticidal activity. The compositions described herein may comprise other agents for pest control, such as microbial extracts, plant growth activators, and/or plant defense agents.

Compositions in some embodiments may comprise one or more chemical acaricides, insecticides, and/or nematicides. Non-limiting examples of chemical acaricides, insecticides, and/or nematicides may include one or more carbamates, diamides, macrocyclic lactones, neonicotinoids, organophosphates, phenylpyrazoles, pyrethrins, spinosyns, synthetic pyrethroids, tetronic acids and/or tetramic acids. Non-limiting examples of chemical acaricides, insecticides and nematicides that can be useful in compositions of the present disclosure include abamectin, acrinathrin, aldicarb, aldoxycarb, alpha-cypermethrin, betacyfluthrin, bifenthrin, cyhalothrin, cypermethrin, deltamethrin, csfenvalcrate, etofenprox, fenpropathrin, fenvalerate, flucythrinate, fosthiazate, lambda-cyhalothrin, gamma-cyhalothrin, permethrin, tau-fluvalinate, transfluthrin, zeta-cypermethrin, cyfluthrin, bifenthrin, tefluthrin, eflusilanat, fubfenprox, pyrethrin, resmethrin, imidacloprid, acetamiprid, thiamethoxam, nitenpyram, thiacloprid, dinotefuran, clothianidin, imidaclothiz, chlorfluazuron, diflubenzuron, lufenuron, teflubenzuron, triflumuron, novaluron, flufenoxuron, hexaflumuron, bistrifluoron, noviflumuron, buprofezin, cyromazine, methoxyfenozide, tebufenozide, halofenozide, chromafenozide, endosulfan, fipronil, ethiprole, pyrafluprole, pyriprole, flubendiamide, chlorantraniliprole (e.g., Rynaxypyr), cyazypyr, emamectin, emamectin benzoate, abamectin, ivermectin, milbemectin, lepimectin, tebufenpyrad, fenpyroximate, pyridaben, fenazaquin, pyrimidifen, tolfenpyrad, dicofol, cyenopyrafen, cyflumetofen, acequinocyl, fluacrypyrin, bifenazate, diafenthiuron, etoxazole, clofentezine, spinosad, triarathen, tetradifon, propargite, hexythiazox, bromopropylate, chinomethionat, amitraz, pyrifluquinazon, pymetrozine, flonicamid, pyriproxyfen, diofenolan, chlorfenapyr, metaflumizone, indoxacarb, chlorpyrifos, spirodiclofen, spiromesifen, spirotetramat, pyridalyl, spinctoram, acephate, triazophos, profenofos, oxamyl, spinetoram, fenamiphos, fenamipclothiahos, 4-{[(6-chloropyrid-3-yl)methyl](2,2-difluoroethyl)amino}furan-2(5H)-one, 3,5-disubstituted-1,2,4-oxadiazole compounds, 3-phenyl-5-(thien-2-yl)-1,2,4-oxadiazole, cadusaphos, carbaryl, carbofuran, ethoprophos, thiodicarb, aldicarb, aldoxycarb, metamidophos, methiocarb, sulfoxaflor, cyantraniliprole and tioxazofen and combinations thereof. Additional non-limiting examples of chemical acaricides, insecticides, and/or nematicides may include one or more of abamectin, aldicarb, aldoxycarb, bifenthrin, carbofuran, chlorantraniliporle, chlothianidin, cyfluthrin, cyhalothrin, cypermethrin, cyantraniliprole, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, spinetoram, spinosad, spirodichlofen, spirotetramat, tefluthrin, thiacloprid, thiamethoxam and/or thiodicarb, and combinations thereof.

Additional non-limiting examples of acaricides, insecticides and nematicides that may be included or used in compositions in some embodiments may be found in Steffey and Gray, Managing Insect Pests, Illinois Agronomy Handbook (2008); and Niblack, Nematodes, Illinois Agronomy Handbook (2008), the contents and disclosures of which are incorporated herein by reference. Non-limiting examples of commercial insecticides which may be suitable for the compositions disclosed herein include CRUISER (Syngenta, Wilmington, Delware), GAUCHO and PONCHO (Gustafson, Plano, Tex.). Active ingredients in these and other commercial insecticides may include thiamethoxam, clothianidin, and imidacloprid. Commercial acaricides, insecticides, and/or nematicides may be used in accordance with a manufacturer's recommended amounts or concentrations.

According to some embodiments, compositions in some embodiments may comprise one or more biopesticidal microorganisms, the presence and/or output of which is toxic to an acarid, insect and/or nematode. For example, the compositions described herein may comprise one or more of Bacillus firmus 1-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43, and/or Metarhizium anisopliae BIO-1020, TAE-001; deposited as DSM 3884, DSM 3885, ATCC 90448, SD 170 and ARSEF 7711), Paecilomyces fumosoroseus FE991, and combinations thereof

Compositions in some embodiments comprise one or more chemical fungicides. Non-limiting examples of chemical fungicides may include one or more aromatic hydrocarbons, benzthiadiazole, carboxylic acid amides, morpholines, phenylamides, phosphonates, thiazolidines, thiophene, quinone outside inhibitors and strobilurins, such as azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester, and 2-(2-(3-(2,6-dichlorophenyl)-1-methyl-allylideneaminooxymethyl)-phenyl)-2-methoxyimino-N-methyl-acetamide, carboxamides, such as carboxanilides (e.g., benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyrazole-4-carboxamide, N-(2-(1,3,3-trimethylbutyl)-phenyl)-1,3-dimethyl-5-fluoro-1H-pyrazole-4-carboxamide), carboxylic morpholides (e.g., dimethomorph, flumorph, pyrimorph), benzoic acid amides (e.g., flumetover, fluopicolide, fluopyram, zoxamide), carpropamid, dicyclomet, mandiproamid, oxytetracyclin, silthiofam, and N-(6-methoxy-pyridin-3-yl) cyclopropanecarboxylic acid amide, azoles, such as triazoles (e.g., azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole) and imidazoles (e.g., cyazofamid, imazalil, pefurazoate, prochloraz, triflumizol); heterocyclic compounds, such as pyridines (e.g., fluazinam, pyrifenox (cf. D1b), 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine), pyrimidines (e.g., bupirimate, cyprodinil, diflumetorim, fenarimol, ferimzone, mepanipyrim, nitrapyrin, nuarimol, pyrimethanil), piperazines (e.g., triforine), pirroles (e.g., fenpiclonil, fludioxonil), morpholines (e.g., aldimorph, dodemorph, dodemorph-acetate, fenpropimorph, tridemorph), piperidines (e.g., fenpropidin); dicarboximides (e.g., fluoroimid, iprodione, procymidone, vinclozolin), non-aromatic 5-membered heterocycles (e.g., famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester), acibenzolar-S-methyl, ametoctradin, amisulbrom, anilazin, blasticidin-S, captafol, captan, chinomethionat, dazomet, debacarb, diclomezine, difenzoquat, difenzoquat-methyl sulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole and 5-chloro-7-(4-methylpiperidin-1-yl)-6-(2,4,6-trifluorophenyl)-[1,2,4]triazolo-[1,5-a]pyrimidine; benzimidazoles, such as carbendazim; and other active substances, such as guanidines (e.g., guanidine, dodine, dodine free base, guazatine, guazatine-acetate, iminoctadine), iminoctadine-triacetate and iminoctadine-tris(albesilate); antibiotics (e.g., kasugamycin, kasugamycin hydrochloride-hydrate, streptomycin, polyoxine and validamycin A), nitrophenyl derivates (e.g., binapacryl, dicloran, dinobuton, dinocap, nitrothal-isopropyl, tecnazen). organometal compounds (e.g., fentin salts, such as fentin-acetate, fentin chloride, fentin hydroxide); sulfur-containing heterocyclyl compounds (e.g., dithianon, isoprothiolane), organophosphorus compounds (e.g., edifenphos, fosetyl, fosetyl-aluminum, iprobenfos, phosphorus acid and its salts, pyrazophos, tolclofos-methyl), organochlorine compounds (e.g., chlorothalonil, dichlofluanid, dichlorophen, flusulfamide, hexachlorobenzene, pencycuron, pentachlorphenole and its salts, phthalide, quintozene, thiophanate-methyl, thiophanates, tolylfluanid, N-(4-chloro-2-nitro-phenyl)-N-ethyl-4-methyl-benzenesulfonamide) and inorganic active substances (e.g., Bordeaux mixture, copper acetate, copper hydroxide, copper oxychloride, basic copper sulfate, sulfur) and combinations thereof. In an aspect, compositions in some embodiments comprise acibenzolar-S-methyl, azoxystrobin, benalaxyl, bixafen, boscalid, carbendazim, cyproconazole, dimethomorph, epoxiconazole, fludioxonil, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thiabendazole, thifluzamide, thiophanate, tolclofos-methyl, trifloxystrobin and triticonazole, and combinations thereof.

For additional examples of fungicides that may be included in compositions in some embodiments see, e.g., Bradley, Managing Diseases, Illinois Agronomy Handbook (2008), the content and disclosure of which are incorporated herein by reference.

Fungicides useful for compositions in some embodiments may exhibit activity against one or more fungal plant pathogens, including but not limited to Phytophthora, Rhizoctonia, Fusarium, Pythium, Phomopsis, Selerotinia or Phakopsora, and combinations thereof. Non-limiting examples of commercial fungicides which may be suitable for the compositions in some embodiments include PROTÉGÉ, RIVAL or ALLEGIANCE FL or LS (Gustafson, Plano, Tex.), WARDEN RTA (Agrilance, St. Paul, Minn.), APRON XL, APRON MAXX RTA or RFC, MAXIM 4FS or XL (Syngenta, Wilmington, Del.), CAPTAN (Arvesta, Guelph, Ontario) and PROTREAT (Nitragin Argentina, Buenos Ares, Argentina). Active ingredients in these and other commercial fungicides include, but are not limited to, fludioxonil, mefenoxam, azoxystrobin and metalaxyl. Commercial fungicides may be used in accordance with a manufacturer's recommended amounts or concentrations.

According to some embodiments, compositions in some embodiments may comprise one or more biopesticidal microorganisms, the presence and/or output of which is toxic to at least one fungus, bacteria, or both. For example, compositions of in some embodiments may comprise one or more of Ampelomyces quisqualis AQ 10® (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens FZB42, Bacillus amyloliquefaciens NRRL B-50349, Bacillus amyloliquefaciens TJ1000 (also known as 1BE, isolate ATCC BAA-390), Bacillus subtilis ATCC 55078, Bacillus subtilis ATCC 55079, Bacillus thuringiensis AQ52 (NRRL B-21619), Candida oleophila 1-182 (e.g., ASPIRE® from Ecogen Inc., USA), Candida saitoana BIOCURE® (in mixture with lysozyme; BASF, USA) and BIOCOAT® (ArystaLife Science, Ltd., Cary, N.C.), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochium dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone Biolnnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANT SHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERMA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma virens GL-21 (SOILGARD®, Certis LLC, USA), Trichoderma virens G1-3 (ATCC 57678), Trichoderma virens G1-21 (Thermo Trilogy Corporation, Wasco, Calif.), Trichoderma virens G1-3 and Bacillus amyloliquefaciens FZB24, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma virens G1-21 and Bacillus amyloliquefaciens NRRL B-50349, Trichoderma virens G1-21 and Bacillus amyloliquefaciens TJ1000, Trichoderma viride TRIECO® (Ecosense Labs. (India) Pvt. Ltd., India, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ), and combinations thereof.

Compositions in some embodiments may comprise one or more chemical herbicides. The herbicides may be a pre-emergent herbicide, a post-emergent herbicide, or a combination thereof. Non-limiting examples of chemical herbicides may comprise one or more acetyl CoA carboxylase (ACCase) inhibitors, acetolactate synthase (ALS) inhibitors, acetanilides, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvylshikimate-3-phosphate (EPSP) synthase inhibitors, glutamine synthetase inhibitors, dihydropteroate synthetase inhibitors, mitosis inhibitors, 4-hydroxyphenyl-pyruvate-dioxygenase (4-HPPD) inhibitors, synthetic auxins, auxin herbicide salts, auxin transport inhibitors, nucleic acid inhibitors and/or one or more salts, esters, racemic mixtures and/or resolved isomers thereof. Non-limiting examples of chemical herbicides that can be useful in compositions of the present disclosure include 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluro, clethodim, clodinafop, clomazone, cyanazine, cycloxydim, cyhalofop, desmedipham, desmetryn, dicamba, diclofop, dimefuron, diuron, dithiopyr, fenoxaprop, fluazifop, fluazifop-P, fluometuron, flufenpyr-ethyl, flumiclorac, flumiclorac-pentyl, flumioxazin, fluoroglycofen, fluthiacet-methyl, fomesafen, fomesafen, glyphosate, glufosinate, halosulfuron, haloxyfop, hexazinone, imazamox, imazaquin, imazethapyr, ioxynil, isoproturon, isoxaflutole, lactofen, linuron, mecoprop, mecoprop-P, mesotrion, metamitron, metazochlor, methibenzuron, metolachlor (and S-metolachlor), metoxuron, metribuzin, monolinuron, oxadiargyl, oxadiazon, oxyfluorfen, phenmedipham, pretilachlor, profoxydim, prometon, prometry, propachlor, propanil, propaquizafop, propisochlor, pyraflufen-ethyl, pyrazon, pyrazolynate, pyrazoxyfen, pyridate, quizalofop, quizalofop-P (e.g., quizalofop-ethyl, quizalofop-P-ethyl, clodinafop-propargyl, cyhalofop-butyl, diclofop-methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins described in U.S. Pat. No. 7,989,393), thenylchlor, tralkoxydim, triclopyr, trietazine, trifloxysulfuron, tropramezone, salts and esters thereof; racemic mixtures and resolved isomers thereof and combinations thereof. In an embodiment, compositions comprise acetochlor, clethodim, dicamba, flumioxazin, fomesafen, glyphosate, glufosinate, mesotrione, quizalofop, saflufenacil, sulcotrione, S-3100 and/or 2,4-D, and combinations thereof.

Additional examples of herbicides that may be included in compositions in some embodiments may be found in Hager, Weed Management, Illinois Agronomy Handbook (2008); and Loux et al., Weed Control Guide for Ohio, Indiana and Illinois (2015), the contents and disclosures of which are incorporated herein by reference. Commercial herbicides may be used in accordance with a manufacturer's recommended amounts or concentrations.

Compositions in some embodiments may comprise one or more virucides.

According to some embodiments, compositions in some embodiments may comprise one or more biopesticidal or herbicidal microorganisms, the presence and/or output of which is toxic to at least one insect, plant (weed), or phytopathogenic virus, as the case may be.

Additional examples of biopesticides that may be included or used in compositions in some embodiments may be found in Burges, supra; Hall & Menn, Biopesticides: Use and Delivery (Humana Press) (1998); McCoy et al., Entomogenous fungi, in CRC Handbook of Natural Pesticides. Microbial Pesticides, Part A. Entomogenous Protozoa and Fungi (C. M. Inoffo, ed.), Vol. 5:151-236 (1988); Samson et al., Atlas of Entomopathogenic Fungi (Springer-Verlag, Berlin) (1988); and deFaria and Wraight, Mycoinsecticides and Mycoacaricides: A comprehensive list with worldwide coverage and international classification of formulation types, Biol. Control (2007), the contents and disclosures of which are incorporated herein by reference. In certain embodiments, a biocontrol microbe may comprise a bacterium of the genus Actinomycetes, Agrobacterium, Arthrobacter, Alcaligenes, Aureobacterium, Azobacter, Bacillus, Beijerinckia, Brevibacillus, Burkholderia, Chromobacterium, Clostridium, Clavibacter, Comamonas, Corynebacterium, Curtobacterium, Enterobacter, Flavobacterium, Gluconobacter, Hydrogenophaga, Klebsiella, Methylobacterium, Paenibacillus, Pasteuria, Photorhabdus, Phyllobacterium, Pseudomonas, Rhizobium, Serratia, Sphingobacterium, Stenotrophomonas, Variovorax, and Xenorhabdus, or any combination thereof. According to some embodiments, a biopesticidal microbe may include one or more of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens. According to some embodiments, a biopesticidal microbe may comprise a fungus of the genus Alternaria, Ampelomyces, Aspergillus, Aureobasidium, Beauveria, Colletotrichum, Coniothyrium, Gliocladium, Metarhizium, Muscodor, Paecilomyces, Trichoderma, Typhula, Ulocladium, and Verticillium. In another aspect a fungus is Beauveria bassiana, Coniothyrium minitans, Gliocladium vixens, Muscodor albus, Paecilomyces lilacinus, or Trichoderma polysporum.

A composition in some embodiments may comprise one or more biocidal agents. A biocidal component may be included or used to prevent fungal and/or bacterial growth in the composition, particularly when the composition is placed in storage. Examples of biocidal agents include dichlorophen or benzyl alcohol hemiformal based compounds, benzoisothiazolinones and rhamnolipids. Non-limiting examples of commercially available biocidal agents include ACTICIDE (THOR), PROXEL (Arch Chemical), and ZONIX (Jeneil).

In addition to a microbial strain or isolate compositions and formulations in some embodiments may further comprise one or more agriculturally beneficial agents, such as biostimulants, nutrients, plant signal molecules, or biologically active agents.

According to some embodiments, compositions may comprise one or more beneficial biostimulants. Biostimulants may enhance metabolic or physiological processes such as respiration, photosynthesis, nucleic acid uptake, ion uptake, nutrient delivery, or a combination thereof. Non-limiting examples of biostimulants that may be included or used in the compositions described herein may include seaweed extracts (e.g., ascophyllum nodosum), bacterial extracts (e.g., extracts of one or more diazotrophs, phosphate-solubilizing microorganisms and/or biopesticides), fungal extracts, humic acids (e.g., potassium humate), fulvic acids, myo-inositol, and/or glycine, and any combinations thereof. According to some embodiments, the biostimulants may comprise one or more Azospirillum extracts (e.g., an extract of media comprising A. brasilense INTA Az-39), one or more Bradyrhizobium extracts (e.g., an extract of media comprising B. elkanii SEMIA 501, B. elkanii SEMIA 587, B. elkanii SEMIA 5019, B. japonicum NRRL B-50586 (also deposited as NRRL B-59565), B. japonicum NRRL B-50587 (also deposited as NRRL B-59566), B. japonicum NRRL B-50588 (also deposited as NRRL B-59567), B. japonicum NRRL B-50589 (also deposited as NRRL B-59568), B. japonicum NRRL B-50590 (also deposited as NRRL B-59569), B. japonicum NRRL B-50591 (also deposited as NRRL B-59570), B. japonicum NRRL B-50592 (also deposited as NRRL B-59571), B. japonicum NRRL B-50593 (also deposited as NRRL B-59572), B. japonicum NRRL B-50594 (also deposited as NRRL B-50493), B. japonicum NRRL B-50608, B. japonicum NRRL B-50609, B. japonicum NRRL B-50610, B. japonicum NRRL B-50611, B. japonicum NRRL B-50612, B. japonicum NRRL B-50726, B. japonicum NRRL B-50727, B. japonicum NRRL B-50728, B. japonicum NRRL B-50729, B. japonicum NRRL B-50730, B. japonicum SEMIA 566, B. japonicum SEMIA 5079, B. japonicum SEMIA 5080, B. japonicum USDA 6, B. japonicum USDA 110, B. japonicum USDA 122, B. japonicum USDA 123, B. japonicum USDA 127, B. japonicum USDA 129 and/or B. japonicum USDA 532C), one or more Rhizobium extracts (e.g., an extract of media comprising R. leguminosarum S012A-2), one or more Sinorhizobium extracts (e.g., an extract of media comprising S. fredii CCBAU114 and/or S. fredii USDA 205), one or more Penicillium extracts (e.g., an extract of media comprising P. bilaiae ATCC 18309, P. bilaiae ATCC 20851, P. bilaiae ATCC 22348, P. bilaiae NRRL 50162, P. bilaiae NRRL 50169, P. bilaiae NRRL 50776, P. bilaiae NRRL 50777, P. bilaiae NRRL 50778, P. bilaiae NRRL 50777, P. bilaiae NRRL 50778, P. bilaiae NRRL 50779, P. bilaiae NRRL 50780, P. bilaiae NRRL 50781, P. bilaiae NRRL 50782, P. bilaiae NRRL 50783, P. bilaiae NRRL 50784, P. bilaiae NRRL 50785, P. bilaiae NRRL 50786, P. bilaiae NRRL 50787, P. bilaiae NRRL 50788, P. bilaiae RS7B-SD1, P. brevicompactum AgRF18, P. canescens ATCC 10419, P. expansum ATCC 24692, P. expansum YT02, P. fellatanum ATCC 48694, P. gaestrivorus NRRL 50170, P. glabrum DAOM 239074, P. glabrum CBS 229.28, P. janthinellum ATCC 10455, P. lanosocoeruleum ATCC 48919, P. radicum ATCC 201836, P. radicum FRR 4717, P. radicum FRR 4719, P. radicum N93/47267 and/or P. raistrickii ATCC 10490), one or more Pseudomonas extracts (e.g., an extract of media comprising P. jessenii PS06), one or more acaricidal, insecticidal and/or nematicidal extracts (e.g., an extract of media comprising Bacillus firmus 1-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43 and Metarhizium anisopliae BIO-1020, TAE-001; deposited as DSM 3884, DSM 3885, ATCC 90448, SD 170 and ARSEF 7711) and/or Paecilomyces fumosoroseus FE991), and/or one or more fungicidal extracts (e.g., an extract of media comprising Ampelomyces quisqualis AQ 10® (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens NRRL B-50349, Bacillus amyloliquefaciens TJ1000 (also known as 1BE, isolate ATCC BAA-390), Bacillus thuringiensis AQ52 (NRRL B-21619), Candida oleophila 1-82 (e.g., ASPIRE® from Ecogen Inc., USA), Candida saitoana BIOCURE® (in mixture with lysozyme; BASF, USA) and BIOCOAT® (ArystaLife Science, Ltd., Cary, N.C.), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochium dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone Biolnnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANTSHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERMA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma virens GL-21 (SOILGARD®, Certis LLC, USA), Trichoderma virens G1-3, ATCC 57678, Trichoderma virens G1-21 (Thermo Trilogy Corporation, Wasco, Calif.), Trichoderma virens G1-3 and Bacillus amyloliquefaciens FZB2, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma virens G1-21 and Bacillus amyloliquefaciens NRRL B-50349, Trichoderma virens G1-21 and Bacillus amyloliquefaciens TJ1000, Trichoderma viride TRIECO® (Ecosense Labs. (India) Pvt. Ltd., Indien, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ)), and combinations thereof

Compositions in some embodiments may comprise one or more biologically active ingredients. Non-limiting examples of biologically active ingredients include plant growth regulators, plant signal molecules, growth enhancers, microbial stimulating molecules, biomolecules, soil amendments, nutrients, plant nutrient enhancers, etc., such as lipochitooligosaccharides (LCO), chitooligosaccharides (CO), chitinous compounds, flavonoids, jasmonic acid or derivatives thereof (e.g., jasmonates), cytokinins, auxins, gibberellins, absiscic acid, ethylene, brassinosteroids, salicylates, macro- and micronutrients, linoleic acid or derivatives thereof, linolenic acid or derivatives thereof, karrikins, etc.) and beneficial microorganisms (e.g., Rhizobium spp., Bradyrhizobium spp., Sinorhizobium spp., Azorhizobium spp., Glomus spp., Gigaspora spp., Hymenoscyphous spp., Oidiodendron spp., Laccaria spp., Pisolithus spp., Rhizopogon spp., Scleroderma spp., Rhizoctonia spp., Acinetobacter spp., Arthrobacter spp, Arthrobotrys spp., Aspergillus spp., Azospirillum spp, Bacillus spp, Burkholderia spp., Candida spp., Chryseomonas spp., Enterobacter spp., Eupenicillium spp., Exiguobacterium spp., Klebsiella spp., Kluyvera spp., Microbacterium spp., Mucor spp., Paecilomyces spp., Paenibacillus spp., Penicillium spp., Pseudomonas spp., Serratia spp., Stenotrophomonas spp., Streptomyces spp., Streptosporangium spp., Swaminathania spp., Thiobacillus spp., Torulospora spp., Vibrio spp., Xanthobacter spp., Xanthomonas spp., etc.), and combinations thereof.

Compositions in some embodiments may comprise one or more lipochitooligosaccharides (LCOs), chitooligosaccharides (COs), and/or chitinous compounds. LCOs, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end. As understood in the art, LCOs differ in the number of GlcNAc residues in the backbone, in the length and degree of saturation of the fatty acyl chain and in the substitutions of reducing and non-reducing sugar residues. See, e.g., Denarie et al., Ann. Rev. Biochem. 65:503 (1996); Diaz et al., Mol. Plant-Microbe Interactions 13:268 (2000); Hungria et al., Soil Biol. Biochem. 29:819 (1997); Hamel et al., Planta 232:787 (2010); and Prome et al., Pure & Appl. Chem. 70(1):55 (1998), the contents and disclosures of which are incorporated herein by reference.

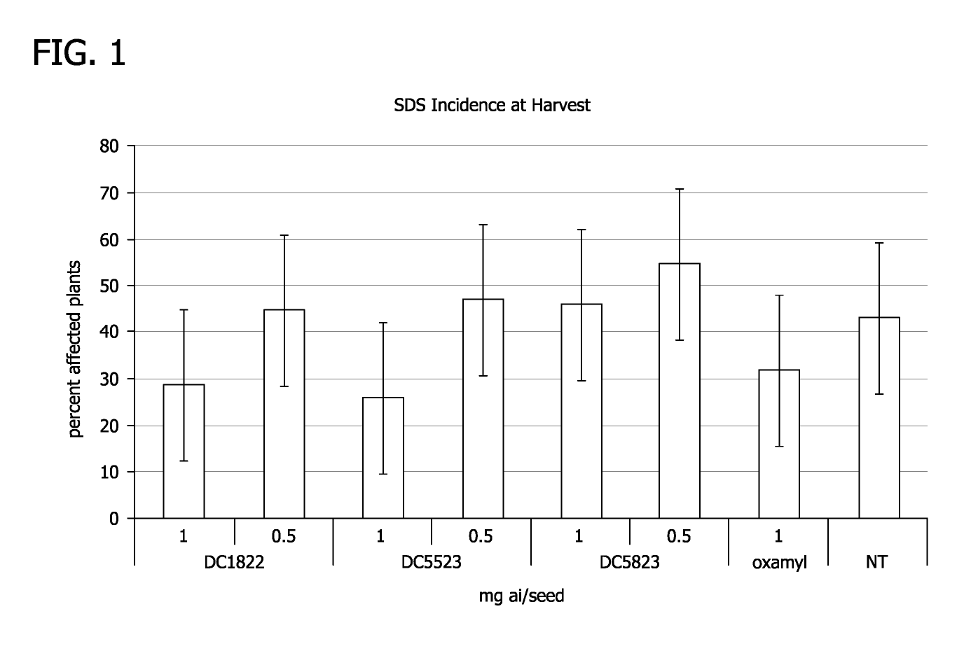

LCOs may be synthetic or obtained from any suitable source. See, e.g., WO 2005/063784, WO 2007/117500 and WO 2008/071674, the contents and disclosures of which are incorporated herein by reference. In some aspects, a synthetic LCO may have the basic structure of a naturally occurring LCO but contains one or more modifications or substitutions, such as those described in Spaink, Crit. Rev. Plant Sci. 54:257 (2000). LCOs and precursors for the construction of LCOs (e.g., COs, which may themselves be useful as a biologically active ingredient) can be synthesized by genetically engineered organisms. See, e.g., Samain et al., Carbohydrate Res. 302:35 (1997); Cottaz et al., Meth. Eng. 7(4):311 (2005); and Samain et al., J. Biotechnol. 72:33 (1999) (e.g., FIG. 1 therein, which shows structures of COs that can be made recombinantly in E. coli harboring different combinations of genes nodBCHL), the contents and disclosures of which are incorporated herein by reference.

LCOs (and derivatives thereof) may be included or utilized in the compositions described herein in various forms of purity and can be used alone or in the form of a culture of LCO-producing bacteria or fungi. For example, OPTIMIZE® (commercially available from Monsanto Company (St. Louis, Mo.)) contains a culture of Bradyrhizobium japonicum that produces LCO. Methods to provide substantially pure LCOs include removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC and the purified LCO molecules can be freeze-dried for long-term storage. According to some embodiments, the LCO(s) included in compositions of the present disclosure is/are at least 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% pure. Compositions and methods in some embodiments may comprise analogues, derivatives, hydrates, isomers, salts and/or solvates of LCOs. LCOs may be incorporated into compositions of the present disclosure in any suitable amount(s)/concentration(s). For example, compositions of the present disclosure comprise about 1×10⁻²⁰ M to about 1×10⁻¹ M LCO(s). For example, compositions of the present disclosure can comprise about 1×10⁻²⁰ M, 1×10⁻¹⁹M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻²M, 1×10⁻¹ M of one or more LCOs. In an aspect, the LCO concentration is 1×10⁻¹⁴ M to 1×10⁻⁵M, 1×10⁻¹²M to 1×10⁻⁶M, or 1×10⁻¹⁰ M to 1×10⁻⁷M. In an aspect, the LCO concentration is 1×10⁻¹⁴M to 1×10⁻⁵M, 1×10⁻¹²M to 1×10⁻⁶M, or 1×10⁻¹⁰ M to 1×10⁻⁷M. The amount/concentration of LCO may be an amount effective to impart a positive trait or benefit to a plant, such as to enhance the disease resistance, growth and/or yield of the plant to which the composition is applied. According to some embodiments, the LCO amount/concentration is not effective to enhance the yield of the plant without beneficial contributions from one or more other constituents of the composition, such as CO and/or one or more pesticides.

Compositions in some embodiments may comprise any suitable COs, perhaps in combination with one or more LCOs. COs differ from LCOs in that they lack the pendant fatty acid chain that is characteristic of LCOs. COs, sometimes referred to as N-acetylchitooligosaccharides, are also composed of GlcNAc residues but have side chain decorations that make them different from chitin molecules [(C₈H₁₃NO₅)ₙ, CAS No. 1398-61-4] and chitosan molecules [(C₅H₁₁NO₄)ₙ, CAS No. 9012-76-4]. See, e.g., D'Haeze et al., Glycobiol. 12(6):79R (2002); Demont-Caulet et al., Plant Physiol. 120(1):83 (1999); Hanel et al., Planta 232:787 (2010); Muller et al., Plant Physiol. 124:733 (2000); Robina et al., Tetrahedron 58:521-530 (2002); Rouge et al., Docking of Chitin Oligomers and Nod Factors on Lectin Domains of the LysM-RLK Receptors in the Medicago-Rhizobium Symbiosis, in The Molecular Immunology of Complex Carbohydrates-3 (Springer Science, 2011); Van der Holst et al., Curr. Opin. Struc. Biol. 11:608 (2001); and Wan et al., Plant Cell 21:1053 (2009), the contents and disclosures of which are incorporated by reference. COs may be obtained from any suitable source. For example, the CO may be derived from an LCO. For example, in an aspect, compositions comprise one or more COs derived from an LCO obtained (i.e., isolated and/or purified) from a strain of Azorhizobium, Bradyrhizobium (e.g., B. japonicum), Mesorhizobium, Rhizobium (e.g., R. leguminosarum), Sinorhizobium (e.g., S. meliloti), or mycorhizzal fungi (e.g., Glomus intraradicus). Alternatively, the CO may be synthetic. Methods for the preparation of recombinant COs are known in the art. See, e.g., Cottaz et al., Meth. Eng. 7(4):311 (2005); Samain et al., Carbohydrate Res. 302:35 (1997); and Samain et al., J. Biotechnol. 72:33 (1999), the contents and disclosures of which are incorporated herein by reference.

COs (and derivatives thereof) may be included or utilized in the compositions described herein in various forms of purity and can be used alone or in the form of a culture of CO-producing bacteria or fungi. According to some embodiments, the CO(s) included in compositions may be at least 0.1%, 0.5%, 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, 99.5% or more pure. It is to be understood that compositions and methods of the present disclosure can comprise hydrates, isomers, salts and/or solvates of COs. COs in some embodiments may be incorporated into compositions in any suitable amount(s)/concentration(s). For example, compositions in some embodiments may comprise about 1×10⁻²⁰ M to about 1×10⁻¹ M COs, such as about 1×10⁻²⁰ M, 1×10⁻¹⁹ M, 1×10⁻¹⁸M, 1×10⁻¹⁷ M, 1×10⁻¹⁶M, 1×10⁻¹⁵ M, 1×10⁻¹⁴ M, 1×10⁻¹³M, 1×10⁻¹² M, 1×10⁻¹¹M, 1×10⁻¹⁰ M, 1×10⁻⁹M, 1×10⁻⁸M, 1×10⁻⁷ M, 1×10⁻⁶M, 1×10⁻⁵M, 1×10⁻⁴ M, 1×10⁻³M, 1×10⁻²M, or 1×10⁻¹ M of one or more COs. For example, the CO concentration may be 1×10⁻¹⁴ M to 1×10⁻⁵M, 1×10⁻¹² M to 1×10⁻⁶ M, or 1×10⁻¹⁰ M to 1×10⁻⁷M. The amount/concentration of CO may be an amount effective to impart or confer a positive trait or benefit to a plant, such as to enhance the soil microbial environment, nutrient uptake, or increase the growth and/or yield of the plant to which the composition is applied. Compositions in some embodiments may comprise one or more suitable chitinous compounds, such as, for example, chitin (IUPAC: N-[5-[[3-acetylamino-4,5-dihydroxy-6-(hydroxymethyl)oxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol), and isomers, salts and solvates thereof.

Chitins and chitosans, which are major components of the cell walls of fungi and the exoskeletons of insects and crustaceans, are composed of GlcNAc residues. Chitins and chitosans may be obtained commercially or prepared from insects, crustacean shells, or fungal cell walls. Methods for the preparation of chitin and chitosan are known in the art. See, e.g., U.S. Pat. No. 4,536,207 (preparation from crustacean shells) and U.S. Pat. No. 5,965,545 (preparation from crab shells and hydrolysis of commercial chitosan); and Pochanavanich et al., Lett. Appl. Microbial. 35:17 (2002) (preparation from fungal cell walls).

Deacetylated chitins and chitosans may be obtained that range from less than 35% to greater than 90% deacetylation and cover a broad spectrum of molecular weights, e.g., low molecular weight chitosan oligomers of less than 15 kD and chitin oligomers of 0.5 to 2 kD; “practical grade” chitosan with a molecular weight of about 15 kD; and high molecular weight chitosan of up to 70 kD. Chitin and chitosan compositions formulated for seed treatment are commercially available. Commercial products include, for example, ELEXA® (Plant Defense Boosters, Inc.) and BEYOND™ (Agrihouse, Inc.).

Compositions in some embodiments may comprise one or more suitable flavonoids, including, but not limited to, anthocyanidins, anthoxanthins, chalcones, coumarins, flavanones, flavanonols, flavans and isoflavonoids, as well as analogues, derivatives, hydrates, isomers, polymers, salts and solvates thereof. Flavonoids are phenolic compounds having the general structure of two aromatic rings connected by a three-carbon bridge. Classes of flavonoids are known in the art. See, e.g., Jain et al., J. Plant Biochem. & Biotechnol. 11:1 (2002); and Shaw et al., Environ. Microbiol. 11:1867 (2006), the contents and disclosures of which are incorporated herein by reference. Several flavonoid compounds are commercially available. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, See, e.g. Ralston et al., Plant Physiol. 137:1375 (2005).

According to embodiments, compositions may comprise one or more flavanones, such as one or more of butin, eriodictyol, hesperetin, hesperidin, homoeriodictyol, isosakuranetin, naringenin, naringin, pinocembrin, poncirin, sakuranetin, sakuranin, and/or sterubin, one or more flavanonols, such as dihydrokaempferol and/or taxifolin, one or more flavans, such as one or more flavan-3-ols (e.g., catechin (C), catechin 3-gallate (Cg), epicatechins (EC), epigallocatechin (EGC) epicatechin 3-gallate (ECg), epigallcatechin 3-gallate (EGCg), epiafzelechin, fisetinidol, gallocatechin (GC), gallcatechin 3-gallate (GCg), guibourtinidol, mesquitol, robinetinidol, theaflavin-3-gallate, theaflavin-3′-gallate, theflavin-3,3′-digallate, thearubigin), flavan-4-ols (e.g., apiforol and/or luteoforol) and/or flavan-3,4-diols (e.g., leucocyanidin, leucodelphinidin, leucofisetinidin, leucomalvidin, luecopelargonidin, leucopeonidin, leucorobinetinidin, melacacidin and/or teracacidin) and/or dimers, trimers, oligomers and/or polymers thereof (e.g., one or more proanthocyanidins), one or more isoflavonoids, such as one or more isoflavones or flavonoid derivatives (e.g, biochanin A, daidzein, formononetin, genistein and/or glycitein), isoflavanes (e.g., equol, ionchocarpane and/or laxifloorane), isoflavandiols, isoflavenes (e.g., glabrene, haginin D and/or 2-methoxyjudaicin), coumestans (e.g., coumestrol, plicadin and/or wedelolactone), pterocarpans, roetonoids, neoflavonoids (e.g, calophyllolide, coutareagenin, dalbergichromene, dalbergin, nivetin), and/or pterocarpans (e.g., bitucarpin A, bitucarpin B, erybraedin A, erybraedin B, erythrabyssin II, erthyrabissin-1, erycristagallin, glycinol, glyceollidins, glyceollins, glycyrrhizol, maackiain, medicarpin, morisianine, orientanol, phaseolin, pisatin, striatine, trifolirhizin), and combinations thereof. Flavonoids and their derivatives may be included in compositions in any suitable form, including, but not limited to, polymorphic and crystalline forms. Flavonoids may be included in compositions in any suitable amount(s) or concentration(s). The amount/concentration of a flavonoid(s) may be an amount effective to impart a benefit to a plant, which may be indirectly through activity on soil microorganisms or other means, such as to enhance plant nutrition and/or yield. According to some embodiments, a flavonoid amount/concentration may not be effective to enhance the nutrition or yield of the plant without the beneficial contributions from one or more other ingredients of the composition, such as LCO, CO, and/or one or more pesticides.

Compositions in some embodiments may comprise one or more suitable non-flavonoid nod-gene inducer(s), including, but not limited to, jasmonic acid ([1R-[1α,2β(Z)]]-3-oxo-2-(pentenyl)cyclopentaneacetic acid; JA), linoleic acid ((Z,Z)-9,12-Octadecadienoic acid) and/or linolenic acid ((Z,Z,Z)-9,12,15-octadecatrienoic acid), and analogues, derivatives, hydrates, isomers, polymers, salts and solvates thereof. Jasmonic acid and its methyl ester, methyl jasmonate (MeJA), collectively known as jasmonates, are octadecanoid-based compounds that occur naturally in some plants (e.g., wheat), fungi (e.g., Botryodiplodia theobromas, Gibberella fujikuroi), yeast (e.g., Saccharomyces cerevisiae) and bacteria (e.g., Escherichia coli). Linoleic acid and linolenic acid may be produced in the course of the biosynthesis of jasmonic acid. Jasmonates, linoleic acid and linolenic acid (and their derivatives) are reported to be inducers of nod gene expression or LCO production by rhizobacteria. See, e.g., Mabood et al. Plant Physiol. Biochem. 44(11):759 (2006); Mabood et al., Agr. J. 98(2):289 (2006); Mabood et al., Field Crops Res. 95(2-3):412 (2006); and Mabood & Smith, Linoleic and linolenic acid induce the expression of nod genes in Bradyrhizobium japonicum USDA 3, Plant Biol. (2001).

Derivatives of jasmonic acid, linoleic acid, and linolenic acid that may be included or used in compositions in some embodiments include esters, amides, glycosides and salts thereof. Representative esters are compounds in which the carboxyl group of linoleic acid, linolenic acid, or jasmonic acid has been replaced with a —COR group, where R is an —OR¹ group, in which R¹ is: an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are each independently: a hydrogen; an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic addition, wherein the carboxylic acid is reacted with an alcohol in the presence of a catalytic amount of a mineral acid. Amides may also be prepared by known methods, such as by reacting the carboxylic acid with the appropriate amine in the presence of a coupling agent, such as dicyclohexyl carbodiimide (DCC), under neutral conditions. Suitable salts of linoleic acid, linolenic acid and jasmonic acid include, for example, base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing a solution of linoleic acid, linolenic acid, or jasmonic acid with a solution of the base. The salts may be precipitated from solution and collected by filtration, or may be recovered by other means such as by evaporation of the solvent.

Non-flavonoid nod-gene inducers may be incorporated into compositions in any suitable amount(s)/concentration(s). For example, the amount/concentration of non-flavonoid nod-gene inducers may be an amount effective to impart or confer a positive trait or benefit to a plant, such as to enhance the disease resistance, growth and/or yield of the plant to which the composition is applied. According to some embodiments, the amount/concentration of non-flavonoid nod-gene inducers may not be effective to enhance the growth and/or yield of the plant without beneficial contributions from one or more other ingredients of the composition, such as a LCO, CO and/or one or more pesticides.

Compositions in some embodiments may comprise karrakins, including but not limited to 2H-furo[2,3-α]pyran-2-ones, as well as analogues, derivatives, hydrates, isomers, polymers, salts and solvates thereof. Examples of biologically acceptable salts of karrakins include acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate and p-toluenesulphonic acid. Additional biologically acceptable metal salts may include alkali metal salts, with bases, examples of which include the sodium and potassium salts. Karrakins may be incorporated into the compositions described herein in any suitable amount(s) or concentration(s). For example, the amount/concentration of a karrakin may be an amount or concentration effective to impart or confer a positive trait or benefit to a plant, such as to enhance the disease resistance, growth and/or yield of the plant to which the composition is applied. In an aspect, a karrakin amount/concentration may not be effective to enhance the disease resistance, growth and/or yield of the plant without beneficial contributions from one or more other ingredients of the composition, such as a LCO, CO and/or one or more pesticides.

Methods for Control of Root-Originated Diseases

Also described is a method for control of root-originated diseases in a plant in a location with nematode pressure, the method comprising administering to a plant, a seed, or soil a compound of any of the formulae described herein in any of the compositions described herein.

Also described is a method for control of Fusarium diseases in a plant in a location with nematode pressure, the method comprising administering to a plant, a seed, or soil a compound of any of the formulae described herein in any of the compositions described herein.

Also described is a method for control of soybean sudden death syndrome in a plant in a location with nematode pressure, the method comprising administering to a plant, a seed, or soil a compound of any of the compounds of the formulae described herein in any of the compositions described herein.

In some instances, the root-originated diseases (e.g., Fusarium diseases, including for example, soybean sudden death syndrome) infects a plant and the composition is applied to soil or to the plant. In some instances, the composition is applied to soil before planting. In some instances, the composition is applied to soil after planting. In some instances, the composition is applied to soil using a drip system. In some instances, the composition is applied to soil using a drench system. In some instances, the composition is applied to plant roots or plant foliage (e.g., leaves, stems). In some instances the composition is tilled into the soil or applied in furrow. In some instances, the composition is applied to a seed.

Also described herein is a method of treating a disorder (e.g., an infection or a disease) caused by fungi, (e.g., fungi of the genus Fusarium) in a subject (e.g., a host plant). The method includes administering to the subject an effective amount of a compound of Formula I, Ia, Ib, II, IIa, IIb, III, IIIa, IIIb, IV, IVa, IVb, V, Va, Vb, VI, VIa, VIb, VII, VIIa or VIIb. The compound may be delivered by several means, including pre-planting, post-planting, or as a drench application.

In still another aspect, methods of inhibiting a fungal infection (e.g., an infection by fungi of the genus Fusarium) are provided. Such methods can include contacting the plant (at any stage of growth), seed, or soil with a compound as described herein, e.g., a compound of Formula I, Ia, Ib, II, IIa, IIb, III, IIIa, IIIb, IV, IVa, IVb, V, Va, Vb, VI, VIa, VIb, VII, VIIa or VIIb.

In another aspect, methods of reducing the viability or fecundity or slowing the growth or development or inhibiting the infectivity of a fungus using a compound, e.g., a compound of Formula I, Ia, Ib, II, IIa, IIb, III, IIIa, IIIb, IV, IVa, IVb, V, Va, Vb, VI, VIa, VIb, VII, VIIa or VIIb is provided. Such methods can include contacting the fungus with specific a compound, e.g., a compound of Formula I, Ia, Ib, II, IIa, IIb, III, IIIa, IIIb, IV, IVa, IVb, V, Va, Vb, VI, VIa, VIb, VII, VIIa or VIIb; (c) reducing the viability or fecundity of the fungus.

Also described is a method for reducing the viability, growth, or fecundity of a fungus, the method comprising exposing the fungus to a compound having Formula I, Ia, Ib, II, IIa, IIb, III, IIIa, IIIb, IV, IVa, IVb, V, Va, Vb, VI, VIa, VIb, VII, VIIa or VIIb. Also described is a method of protecting a plant or seed from a fungal infection, the method comprising applying to the plant, to soil, or to the seed of the plant a compound of Formula I, Ia, Ib, II, IIa, IIb, III, Ma, Mb, IV, IVa, IVb, V, Va, Vb, VI, VIa, VIb, VII, VIIa or VIIb.

Described herein are methods for control of root-originated diseases in a plant in a location with nematode pressure (e.g., Fusarium diseases, including for example, soybean sudden death syndrome) by administering a compound described herein. The methods include administering to a plant, a seed, or soil a composition comprising an effective amount of a compound or a mixture of compounds having any of the formulae described herein, for example one of the following formulas:

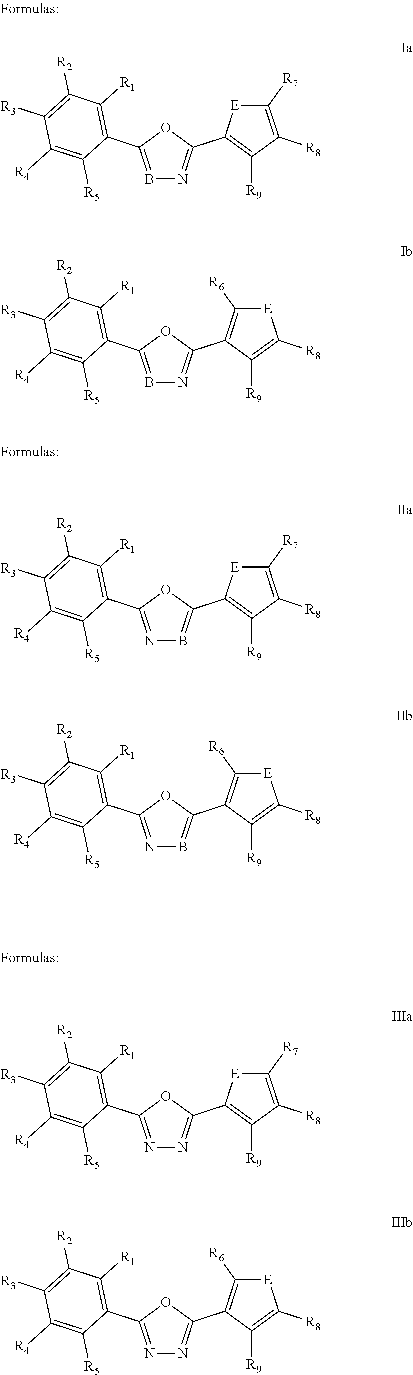

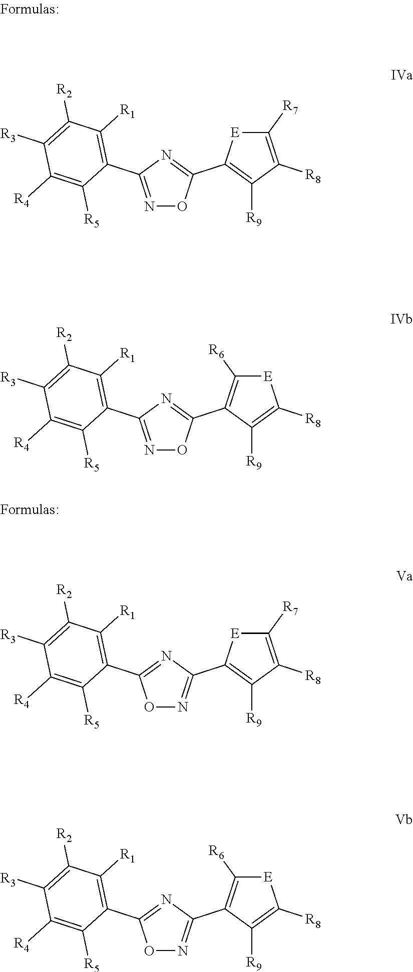

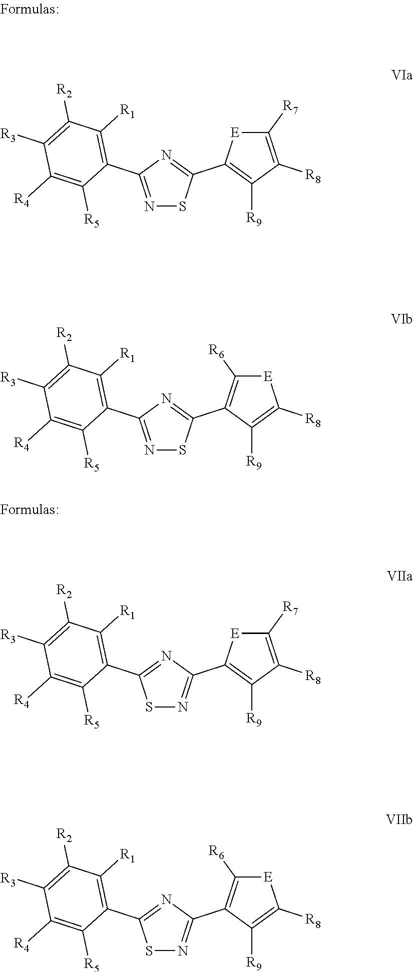

wherein,

R₁ and R₅ are independently selected from hydrogen, CH₃, F, Cl, Br, CF₃, and OCF₃;

R₂ and R₄ are independently selected from hydrogen, F, Cl, Br, and CF₃;

R₃ is selected from hydrogen, CH₃, CF₃, F, Cl, Br, OCF₃, OCH₃, CN, and CO;

R₈ is selected from hydrogen and fluorine;

R₆ and R₉ are independently selected from hydrogen, F, Cl, CH₃, and OCF₃;

B is C(H) or C(CH₃);

E is O or S.

In some cases, R₁ and R₅ are not both H.

Also provided is a method for control of root originated diseases (e.g., Fusarium diseases, including for example, soybean sudden death syndrome) in a plant in a location with nematode pressure comprising administering to a plant, a seed, or soil a composition comprising an effective amount of: (a) a compound selected from the group consisting of 3-phenyl-5-(thiophen-2-yl)-1,2,4-oxadiazole, 3-(4-fluorophenyl)-5-(thiophen-2-yl)-1,2,4-oxadiazole, 3-(4-chlorophenyl)-5-(furan-2-yl)-1,2,4-oxadiazole, 3-(4-chlorophenyl)-5-(thiophen-2-yl)-1,2,4-oxadiazole, 3-(4-chloro-2-methylphenyl)-5-(furan-2-yl)-1,2,4-oxadiazole, 5-(4-chloro-2-methylphenyl)-3-(furan-2-yl)-1,2,4-oxadiazole, 3-(4-bromo-2-methylphenyl)-5-(furan-2-yl)-1,2,4-oxadiazole, 3-(4-fluoro-2-methylphenyl)-5-(thiophen-2-yl)-1,2,4-oxadiazole, difluorophenyl)-5-(thiophen-2-yl)-1,2,4-oxadiazole, 3-(4-bromo-2-fluorophenyl)-5-(thiophen-2-yl)-1,2,4-oxadiazole, 5-(thiophen-2-yl)-3-(2,4,6-trifluorophenyl)-1,2,4-oxadiazole, 3-(2,4-dichlorophenyl)-5-(furan-2-yl)-1,2,4-oxadiazole, 3-(4-bromo-2-chlorophenyl)-5-(furan-2-yl)-1,2,4-oxadiazole, 3-(2-chloro-4-fluorophenyl)-5-(thiophen-2-yl)-1,2,4-oxadiazole, 3-(4-chlorophenyl)-5-(thiophen-2-yl)-1,2,4-thiadiazole, 3-(4-chlorophenyl)-5-(furan-2-yl)-1,2,4-thiadiazole, 3-(4-chlorophenyl)-5-(3-methylfuran-2-yl)-1,2,4-oxadiazole, 5-(4-chloro-2-fluorophenyl)-2-(thiophen-2-yl)oxazole, 2-(4-chloro-2-fluorophenyl)-5-(thiophen-2-yl)oxazole, 5-(4-chloro-2-fluorophenyl)-2-(furan-2-yl)oxazole, 5-(4-chloro-2-methylphenyl)-2-(furan-3-yl)oxazole, 3-(4-chloro-2-methylphenyl)-5-(furan-2-yl)-1,2,4-thiadiazole, 5-(4-chloro-2-methylphenyl)-3-(furan-2-yl)-1,2,4-thiadiazole, 3-(4-bromo-2-methylphenyl)-5-(furan-2-yl)-1,2,4-thiadiazole, 5-(furan-2-yl)-3-(4-methoxy-2-methylphenyl)-1,2,4-oxadiazole, 3-(6-chloropyridin-3-yl)-5-(thiophen-2-yl)-1,2,4-thiadiazole, 3-(6-chloropyridin-3-yl)-5-(furan-2-yl)-1,2,4-thiadiazole, 5-(2,4-difluorophenyl)-2-(thiophen-2-yl)oxazole, 5-(2,4-difluorophenyl)-2-(furan-2-yl)oxazole, 5-(4-bromo-2-fluorophenyl)-2-(thiophen-2-yl)oxazole, 5-(4-bromo-2-fluorophenyl)-2-(furan-2-yl)oxazole, 3-(2,4-difluorophenyl)-5-(furan-2-yl)-1,2,4-thiadiazole, 3-(4-chloro-2-fluorophenyl)-5-(furan-2-yl)-1,2,4-thiadiazole, 3-(4-bromo-2-fluorophenyl)-5-(furan-2-yl)-1,2,4-thiadiazole, 3-(2,4-difluorophenyl)-5-(thiophen-2-yl)-1,2,4-thiadiazole, 3-(4-chloro-2-fluorophenyl)-5-(thiophen-2-yl)-1,2,4-thiadiazole, 3-(4-bromo-2-fluorophenyl)-5-(thiophen-2-yl)-1,2,4-thiadiazole, 5-(furan-2-yl)-3-(4-methoxy-2-methylphenyl)-1,2,4-thiadiazole, 3-(2,4-dichlorophenyl)-5-(furan-2-yl)-1,2,4-thiadiazole, 3-(4-bromo-2-chlorophenyl)-5-(furan-2-yl)-1,2,4-thiadiazole, 3-(2,6-dichloropyridin-3-yl)-5-(furan-2-yl)-1,2,4-thiadiazole, 5-(2,4-dichlorophenyl)-2-(thiophen-2-yl)oxazole, 3-(4-chlorophenyl)-5-(thiophen-3-yl)-1,2,4-oxadiazole, 5-(4-chloro-2-methylphenyl)-2-(furan-3-yl)oxazole.

Also featured is a method for control of soybean sudden death syndrome in a plant in a location with nematode pressure comprising administering to a plant, a seed, or soil a composition comprising an effective amount of: (a) a compound selected from the group consisting of 3-phenyl-5-(thiophen-2-yl)-1,2,4-oxadiazole, 3-(4-fluorophenyl)-5-(thiophen-2-yl)-1,2,4-oxadiazole, 3-(4-chlorophenyl)-5-(furan-2-yl)-1,2,4-oxadiazole, 3-(4-chlorophenyl)-5-(thiophen-2-yl)-1,2,4-oxadiazole, 3-(4-chloro-2-methylphenyl)-5-(furan-2-yl)-1,2,4-oxadiazole, 5-(4-chloro-2-methylphenyl)-3-(furan-2-yl)-1,2,4-oxadiazole, 3-(4-bromo-2-methylphenyl)-5-(furan-2-yl)-1,2,4-oxadiazole, 3-(4-fluoro-2-methylphenyl)-5-(thiophen-2-yl)-1,2,4-oxadiazole, 3-(2,4-difluorophenyl)-5-(thiophen-2-yl)-1,2,4-oxadiazole, 3-(4-bromo-2-fluorophenyl)-5-(thiophen-2-yl)-1,2,4-oxadiazole, 5-(thiophen-2-yl)-3-(2,4,6-trifluorophenyl)-1,2,4-oxadiazole, 3-(2,4-dichlorophenyl)-5-(furan-2-yl)-1,2,4-oxadiazole, 3-(4-bromo-2-chlorophenyl)-5-(furan-2-yl)-1,2,4-oxadiazole, 3-(2-chloro-4-fluorophenyl)-5-(thiophen-2-yl)-1,2,4-oxadiazole, 3-(4-chlorophenyl)-5-(thiophen-2-yl)-1,2,4-thiadiazole, 3-(4-chlorophenyl)-5-(furan-2-yl)-1,2,4-thiadiazole, 3-(4-chlorophenyl)-5-(3-methylfuran-2-yl)-1,2,4-oxadiazole, 5-(4-chloro-2-fluorophenyl)-2-(thiophen-2-yl)oxazole, 2-(4-chloro-2-fluorophenyl)-5-(thiophen-2-yl)oxazole, 5-(4-chloro-2-fluorophenyl)-2-(furan-2-yl)oxazole, 5-(4-chloro-2-methylphenyl)-2-(furan-3-yl)oxazole, 3-(4-chloro-2-methylphenyl)-5-(furan-2-yl)-1,2,4-thiadiazole, 5-(4-chloro-2-methylphenyl)-3-(furan-2-yl)-1,2,4-thiadiazole, 3-(4-bromo-2-methylphenyl)-5-(furan-2-yl)-1,2,4-thiadiazole, 5-(furan-2-yl)-3-(4-methoxy-2-methylphenyl)-1,2,4-oxadiazole, 3-(6-chloropyridin-3-yl)-5-(thiophen-2-yl)-1,2,4-thiadiazole, 3-(6-chloropyridin-3-yl)-5-(furan-2-yl)-1,2,4-thiadiazole, 5-(2,4-difluorophenyl)-2-(thiophen-2-yl)oxazole, 5-(2,4-difluorophenyl)-2-(furan-2-yl)oxazole, 5-(4-bromo-2-fluorophenyl)-2-(thiophen-2-yl)oxazole, 5-(4-bromo-2-fluorophenyl)-2-(furan-2-yl)oxazole, 3-(2,4-difluorophenyl)-5-(furan-2-yl)-1,2,4-thiadiazole, 3-(4-chloro-2-fluorophenyl)-5-(furan-2-yl)-1,2,4-thiadiazole, 3-(4-bromo-2-fluorophenyl)-5-(furan-2-yl)-1,2,4-thiadiazole, 3-(2,4-difluorophenyl)-5-(thiophen-2-yl)-1,2,4-thiadiazole, 3-(4-chloro-2-fluorophenyl)-5-(thiophen-2-yl)-1,2,4-thiadiazole, 3-(4-bromo-2-fluorophenyl)-5-(thiophen-2-yl)-1,2,4-thiadiazole, 5-(furan-2-yl)-3-(4-methoxy-2-methylphenyl)-1,2,4-thiadiazole, 3-(2,4-dichlorophenyl)-5-(furan-2-yl)-1,2,4-thiadiazole, 3-(4-bromo-2-chlorophenyl)-5-(furan-2-yl)-1,2,4-thiadiazole, 3-(2,6-dichloropyridin-3-yl)-5-(furan-2-yl)-1,2,4-thiadiazole, 5-(2,4-dichlorophenyl)-2-(thiophen-2-yl)oxazole, 3-(4-chlorophenyl)-5-(thiophen-3-yl)-1,2,4-oxadiazole, 5-(4-chloro-2-methylphenyl)-2-(furan-3-yl)oxazole.

Also featured is a method for control of root-originated diseases (e.g., Fusarium diseases, including for example, soybean sudden death syndrome) in a plant in a location with nematode pressure comprising administering to a plant, a seed, or soil a composition comprising an effective amount of: (a) a compound selected from the group consisting of 5-(4-bromophenyl)-2-(thiophen-2-yl)oxazole, 2-(2-fluorophenyl)-5-(furan-2-yl)oxazole, 5-(isoxazol-5-yl)-3-(4-(trifluoromethoxy)phenyl)-1,2,4-oxadiazole, 2-phenyl-5-p-tolyl-1,3,4-oxadiazole, 5-(4-fluorophenyl)-2-(thiophen-2-yl)oxazole, 5-(furan-2-yl)-3-p-tolyl-1,2,4-oxadiazole, 2-(4-chlorophenyl)-5-(thiophen-2-yl)oxazole, 2-(3-methoxyphenyl)-5-phenyl-1,3,4-oxadiazole, 5-(4-bromophenyl)-2-(furan-2-yl)oxazole, 5-(4-chlorophenyl)-2-(thiophen-3-yl)oxazole, 2-(furan-2-yl)-5-phenyloxazole, 5-(4-chlorophenyl)-2-(furan-2-yl)oxazole, 5-(furan-2-yl)-3-(4-iodophenyl)-1,2,4-oxadiazole, 5-(furan-2-yl)-3-(oxazol-2-yl)-1,2,4-oxadiazole, 5-(4-propylphenyl)-3-(thiophen-2-yl)-1,2,4-oxadiazole, 2-(4-bromophenyl)-5-(thiophen-2-yl)oxazole, 3-(4-bromophenyl)-5-(furan-2-yl)-1,2,4-oxadiazole, 2-(3-chlorophenyl)-5-(thiophen-2-yl)oxazole.

In certain embodiments of the method, the fungus infects plants and the composition is applied to the soil or to plants; the composition is applied to soil before planting; the composition is applied to soil after planting; the composition is applied to soil using a drip system; the composition is applied to soil using a drench system; the composition is applied to plant roots; the pesticidal composition is applied to seeds; and/or the composition is applied to the foliage of plants.

The compounds may be supplied to plants exogenously, through sprays for example. These compounds may also be applied as a seed coat. The compounds can be applied to plants or the environment of soybean plants needing control of soybean sudden death syndrome. The compositions may be applied by, for example drench or drip techniques. With drip applications compounds can be applied directly to the base of the plants or the soil immediately adjacent to the plants. The composition may be applied through existing drip irrigation systems. Alternatively, a drench application can be used where a sufficient quantity of composition is applied such that it drains to the root area of the plants.

The concentration of the composition should be sufficient to control the fungal pathogen without causing significant phytotoxicity to the desired plant. The compounds disclosed herein have a good therapeutic window.

Tioxazafen (3-phenyl-5-(2-thienyl)-1,2,4-oxadiazole) is a particularly preferred compound for use in accordance with the methods, compositions, and products described herein.

The agents described herein can be applied in conjunction with another pesticidal agents. The second agent may, for example, be applied simultaneously or sequentially.

The methods described herein may also be used to improve one or more agronomic characteristics of desired crop plants. The desired crop plants can be, for example, soybeans, cotton, corn, tobacco, wheat, strawberries, tomatoes, banana, sugar cane, sugar beet, potatoes, or citrus. Non-limiting examples of agronomic characteristics that may be improved include yield, plant stand, crop safety, stalk lodging, plant vigor, and root health. Plant stand refers to the number of plants emerged at a specified time. Plant vigor is a measure of plant growth or foliage volume through time after planting. Crop safety is a measurement of the detrimental impact on a seed caused by various factors including crop protection agents. Yield (also known as “agricultural output”) refers to either the measure of the amount of a crop harvested per unit area of land cultivation or the seed generation of the plant itself.

The compounds, compositions, and methods described herein can be used to prepare treated seeds that are less susceptible to infection by fungi of the genus Fusarium. The compounds, compositions, and methods described herein can be used to prepare treated seeds that are less susceptible to infection by fungi of the genus Pythium. The compounds, compositions, and methods described herein can be used to prepare treated seeds that are less susceptible to infection by fungi of the genus Phytophthora. The compounds, compositions, and methods described herein can be used to prepare treated seeds that are less susceptible to infection by fungi of the genus Rhizoctonia.

Provided herein, for example, is a method of controlling soybean sudden death syndrome, the method comprising administering to a soybean plant, a soybean seed, or soil a composition comprising an effective amount of a compound as described herein (e.g., a compound of Formula I, Ia, Ib, II, IIa, IIb, III, IIIa, IIIb, IV, IVa, IVb, V, Va, Vb, VI, VIa, VIb, VII, VIIa or VIIb), and planting the seed in a location wherein soybean plants are at a significant risk of disease caused by fungi of the genus Fusarium.

Provided herein, for example, is a method of controlling corn disease, the method comprising administering to a corn plant, a corn seed, or soil a composition comprising an effective amount of a compound as described herein (e.g., a compound of Formula I, Ia, Ib, II, IIa, IIb, III, IIIa, IIIb, IV, IVa, IVb, V, Va, Vb, VI, VIa, VIb, VII, VIIa or VIIb), and planting the seed in a location wherein corn plants are at a significant risk of disease caused by fungi of the genus Fusarium.

Provided herein, for example, is a method of controlling cotton disease, the method comprising administering to a cotton plant, a cotton seed, or soil a composition comprising an effective amount of a compound as described herein (e.g., a compound of Formula I, Ia, Ib, II, IIa, IIb, III, IIIa, IIIb, IV, IVa, IVb, V, Va, Vb, VI, VIa, VIb, VII, VIIa or VIIb), and planting the seed in a location wherein cotton plants are at a significant risk of disease caused by fungi of the genus Fusarium.

Treated Seeds

The treated seeds described herein may comprise a compound as described herein in an amount sufficient to reduce the incidence of root-originated diseases in plants. In one embodiment, the treated seeds described herein may comprise a compound as described herein in an amount sufficient to reduce the incidence of root-originated diseases in soy (including Fusarium diseases, including for example soybean death syndrome). For example, treated seeds described herein may comprise a compound as described herein in an amount of at least about 0.1 grams a.i. per gram of seed; at least about 0.2 grams a.i. per gram of seed; at least about 0.25 grams a.i. per gram of seed; at least about 0.3 grams a.i. per gram of seed; at least about 0.375 grams a.i. per gram of seed; at least about 0.5 grams a.i. per gram of seed; or at least about 1 gram a.i. per gram of seed. Treated seeds described herein may comprise a compound as described herein in an amount of from about 0.05 to about 0.5 grams a.i. per gram of seed; from about 0.1 to about 0.5 grams a.i. per gram of seed; from about 0.1 to about 0.4 grams a.i. per gram of seed; or from about 0.2 to about 0.3 grams a.i. per gram of seed.

The treated seeds described herein may comprise a compound as described herein in an amount sufficient to reduce the incidence of root-originated diseases in corn (including Fusarium diseases, including for example, seed rot and seedling blight). For example, treated seeds described herein may comprise a compound as described herein in an amount of at least about 0.1 grams a.i. per gram of seed; at least about 0.2 grams a.i. per gram of seed; at least about 0.25 grams a.i. per gram of seed; at least about 0.3 grams a.i. per gram of seed; at least about 0.375 grams a.i. per gram of seed; at least about 0.5 grams a.i. per gram of seed; or at least about 1 gram a.i. per gram of seed. Treated seeds described herein may comprise a compound as described herein in an amount of from about 0.05 to about 0.5 grams a.i. per gram of seed; from about 0.1 to about 0.5 grams a.i. per gram of seed; from about 0.1 to about 0.4 grams a.i. per gram of seed; or from about 0.2 to about 0.3 grams a.i. per gram of seed.

The treated seeds described herein may comprise a compound as described herein in an amount sufficient to reduce the incidence of root-originated diseases in cotton (including Fusarium diseases, including for example seedling blight and Fusarium wilt). For example, treated seeds described herein may comprise a compound as described herein in an amount of at least about 0.1 grams a.i. per gram of seed; at least about 0.2 grams a.i. per gram of seed; at least about 0.25 grams a.i. per gram of seed; at least about 0.3 grams a.i. per gram of seed; at least about 0.375 grams a.i. per gram of seed; at least about 0.5 grams a.i. per gram of seed; or at least about 1 gram a.i. per gram of seed. Treated seeds described herein may comprise a compound as described herein in an amount of from about 0.05 to about 0.5 grams a.i. per gram of seed; from about 0.1 to about 0.5 grams a.i. per gram of seed; from about 0.1 to about 0.4 grams a.i. per gram of seed; or from about 0.2 to about 0.3 grams a.i. per gram of seed.

### Additional Definitions

The term “halo” or “halogen” refers to any radical of fluorine, chlorine, bromine or iodine.

The term “alkyl” as employed herein by itself or as part of another group refers to both straight and branched chain radicals of up to ten carbons. Typical C₁₋₁₀ alkyl groups include methyl, ethyl, propyl, isopropyl, butyl, sec-butyl, tert-butyl, 3-pentyl, hexyl and octyl groups, which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

Alkoxy groups contain oxygen substituted by one of the C1-10 alkyl groups mentioned above, which may be optionally substituted.

Alkylthio groups contain sulfur substituted by one of the C1-10 alkyl groups mentioned above, which may be optionally substituted. Also included are the sulfoxides and sulfones of such alkylthio groups.

Amino groups include —NH2, —NHR₁₅ and —NR₁₅R₁₆, wherein R₁₅ and R₁₆ are C1-10 alkyl or cycloalkyl groups, or R₁₅ and R₁₆ are combined with the N to form a ring structure, such as a piperidine, or R₁₅ and R₁₆ are combined with the N and other group to form a ring, such as a piperazine. The alkyl group may be optionally substituted.

The term “aryl” as employed herein by itself or as part of another group refers to monocyclic, bicyclic or tricyclic aromatic groups containing from 6 to 14 carbons in the ring.

Common aryl groups include C6-14 aryl, preferably C6-10 aryl. Typical C6-14 aryl groups include phenyl, naphthyl, phenanthrenyl, anthracenyl, indenyl, azulenyl, biphenyl, biphenylenyl and fluorenyl groups.

Cycloalkyl groups are C3-8 cycloalkyl. Typical cycloalkyl groups include cyclopropyl, cyclobutyl, cyclopentyl, cyclohexyl and cycloheptyl.

The term “arylalkyl” is used herein to mean any of the above-mentioned C1-10 alkyl groups substituted by any of the above-mentioned C6-14 aryl groups. Preferably the arylalkyl group is benzyl, phenethyl or naphthylmethyl.

The term “arylalkenyl” is used herein to mean any of the above-mentioned C2-10 alkenyl groups substituted by any of the above-mentioned C6-14 aryl groups.

The term “arylalkynyl” is used herein to mean any of the above-mentioned C2-10 alkynyl groups substituted by any of the above-mentioned C6-14 aryl groups.

The term “aryloxy” is used herein to mean oxygen substituted by one of the above-mentioned C6-14 aryl groups, which may be optionally substituted. Common aryloxy groups include phenoxy and 4-methylphenoxy.

The term “arylalkoxy” is used herein to mean any of the above mentioned C1-10 alkoxy groups substituted by any of the above-mentioned aryl groups, which may be optionally substituted. Example arylalkoxy groups include benzyloxy and phenethyloxy.

Example haloalkyl groups include C1-10 alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Acylamino (acylamido) groups include any C1-6 acyl (alkanoyl) attached to an amino nitrogen, e.g., acetamido, chloroacetamido, propionamido, butanoylamido, pentanoylamido and hexanoylamido, as well as aryl-substituted C1-6 acylamino groups, e.g., benzoylamido, and pentafluorobenzoylamido.

Common acyloxy groups are any C1-6 acyl (alkanoyl) attached to an oxy (—O—) group, e.g., formyloxy, acetoxy, propionoyloxy, butanoyloxy, pentanoyloxy and hexanoyloxy.

The term heterocycle is used herein to mean a saturated or partially saturated 3-7 membered monocyclic, or 7-10 membered bicyclic ring system, which consists of carbon atoms and from one to four heteroatoms independently selected from the group consisting of 0, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable.

Common saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroatoms.

Example heteroaryl groups include thienyl (thiophenyl), benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-α]pyrimidin-4-one, pyrazolo[1,5-α]pyrimidinyl, including without limitation pyrazolo[1,5-α]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The term “heteroaryloxy” is used herein to mean oxygen substituted by one of the above-mentioned heteroaryl groups, which may be optionally substituted. Useful heteroaryloxy groups include pyridyloxy, pyrazinyloxy, pyrrolyloxy, pyrazolyloxy, imidazolyloxy and thiophenyloxy.

The term “heteroarylalkoxy” is used herein to mean any of the above-mentioned C1-10 alkoxy groups substituted by any of the above-mentioned heteroaryl groups, which may be optionally substituted.

A permeation enhancer is generally an agent that facilitates the active compounds described herein.

A co-solvent (i.e., a latent solvent or indirect solvent) is an agent that becomes an effective solvent in the presence of an active solvent and can improve the properties of the primary (active) solvent.

When introducing elements herein, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects are achieved and other advantageous results attained.

As various changes could be made in the above products and methods without departing from the scope of the disclosure, it is intended that all matter contained in the above description shall be interpreted as illustrative and not in a limiting sense.

### EXAMPLES

### Example 1: Description of Synthesis of the Compounds of the Formula I to VII

The compounds of the Formulas I to VII may be prepared using methods known to those skilled in the art. Specifically, the compounds of Formulae Ia and Ib can be prepared as illustrated by the exemplary reaction in Scheme 1. The alpha aminoketones 3 are prepared from the acetophenones 1 in a two-step procedure that involves bromination with 4(-dimethylamino)pyridine tribromide and subsequent amination of the bromide intermediate 2 with sodium diformylamide. The aminoketone 3 is then reacted with an appropriate acyl chloride 4 to yield the acylaminoketone 5. A cyclization of the linear precursor 5 to the 2,5-disubstituted-1,3-oxazole analog 6 is accomplished with phosporousoxychloride in DMF in good yields.

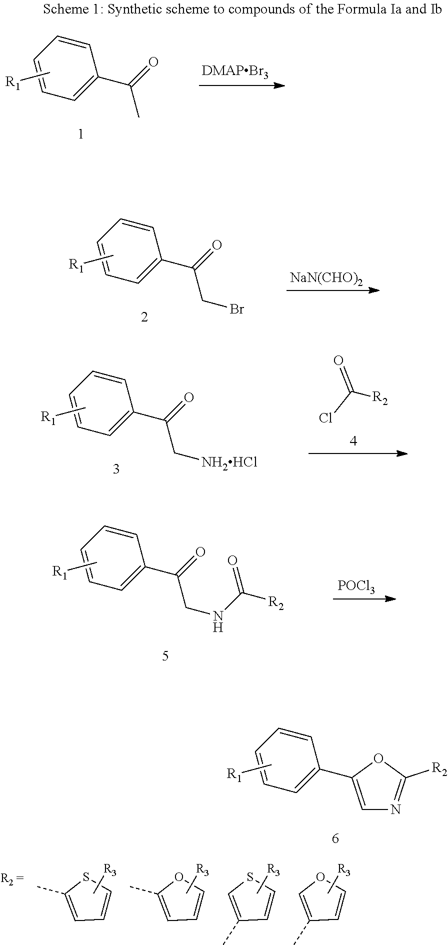

Specifically, the compounds of Formulae IIa and IIb can be prepared as illustrated by the exemplary reaction in Scheme 2. The alpha aminoketone 2 is prepared from the bromide precursor 1 by amination with sodium diformylamide and then reacted with acyl chloride 3 to yield the acylaminoketone 4. A cyclization of the linear precursor 4 to the 2,5-disubstituted-1,3-oxazole analog 5 is accomplished with phosporousoxychloride in DMF in good yields.

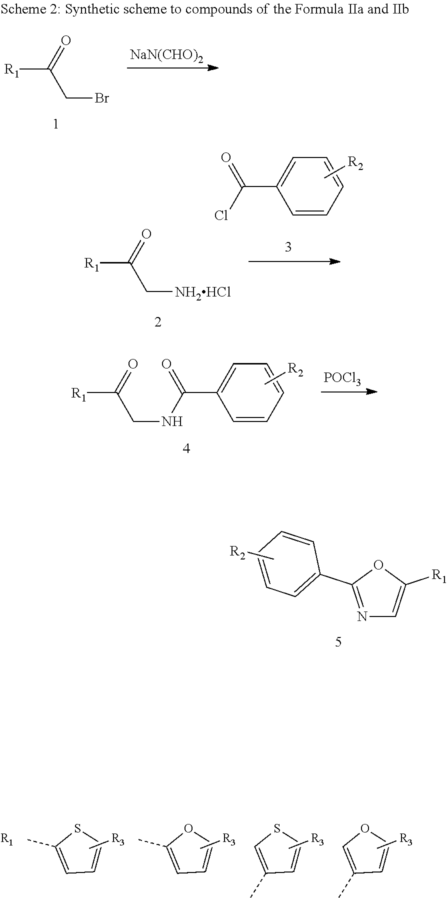

Specifically, the compounds of Formulae Ma and III b can be prepared as illustrated by the exemplary reaction in Scheme 3. The benzohydrazide 1 is reacted with the acyl chloride 2 in chloroform in the presence of trietylamine (TEA) at ambient temperature to give acyl benzohydrazide 3. A cyclization of the diacylhydrazine 3 to the 2,5-disubstituted-1,3,4-oxadiazole compound 4 is accomplished with phosporouschloride (POCl3) in DMF.

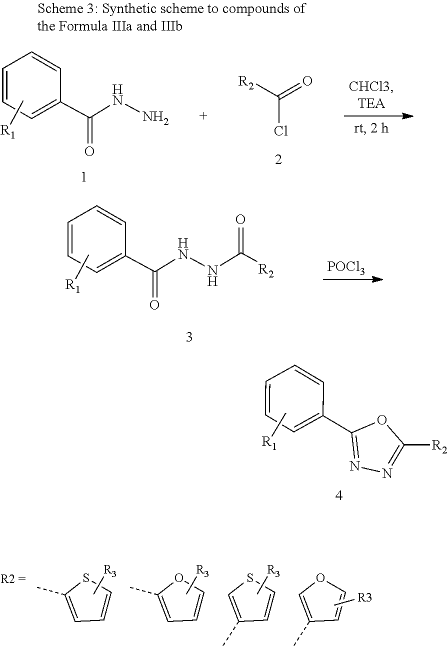

Specifically, the compounds of Formulae IVa and IVb can be prepared as illustrated by the exemplary reaction in Scheme 4. The benzonitrile 1 is converted to the corresponding hydroxyimidate 2 when reacted with hydroxylamine hydrochloride in the presence of DIEA in methanol at room temperature overnight. Then the benzohydroxyimidate 2 is acylated with an appropriate furan or thiophene carbonyl chloride (R2-CO—Y) in the presence of pyridine, followed with DCC dehydration to give the 3,5-disubstituted-1,2,4-oxadiazole product.

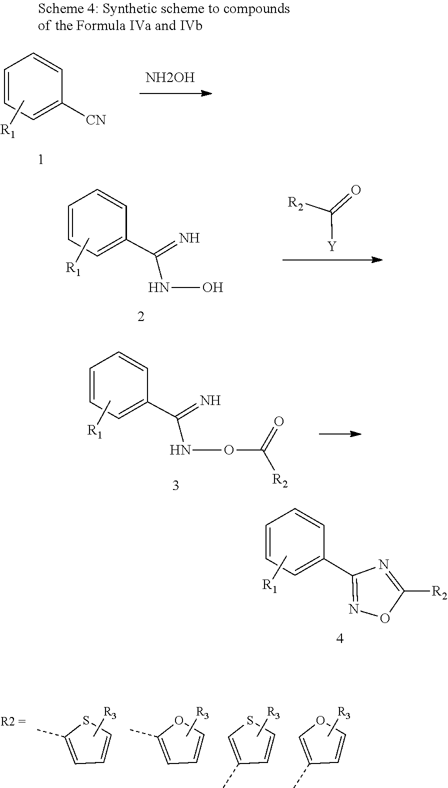

Specifically, the compounds of Formulae Va and Vb can be prepared as illustrated by the exemplary reaction in Scheme 5.

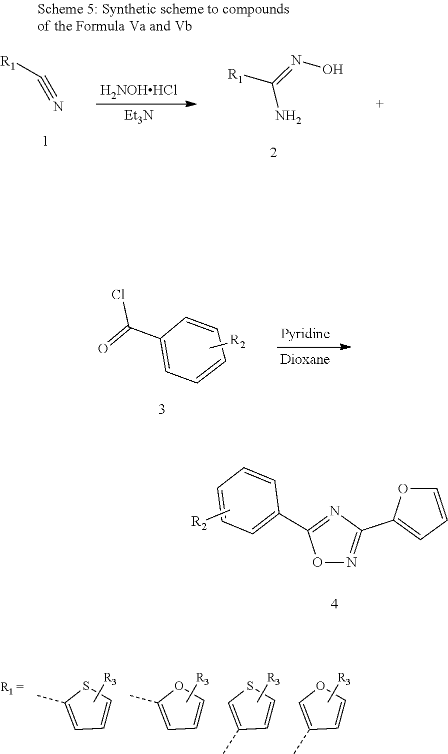

First, the appropriate analog of furan or thiophene nitrile 1 is converted to the corresponding hydroxyimidate 2 by reacting with hydroxylamine in methanol in the presence of DIEA. Then, the intermediate 2 is reacted with the appropriately substituted benzoyl chloride 3 in pyridine-dioxane to give the desired 3,5-disubstituted-1,2,4-oxadiazole product 4.

Specifically, the compounds of Formulae VIa and VIb can be prepared as illustrated by the exemplary reaction in Scheme 6. The synthesis starts with the reaction of an appropriate benzamide substrate 1 with chlorocarbonylsulfenyl chloride to yield the oxathiazolone compound 2. In the next step the oxathiazoline intermediate 2 is reacted with an appropriate furan or thiophene nitrile in toluene under microwave conditions to give the desired 3,5-disubstituted-1,2,4-thiadiazole product 3.

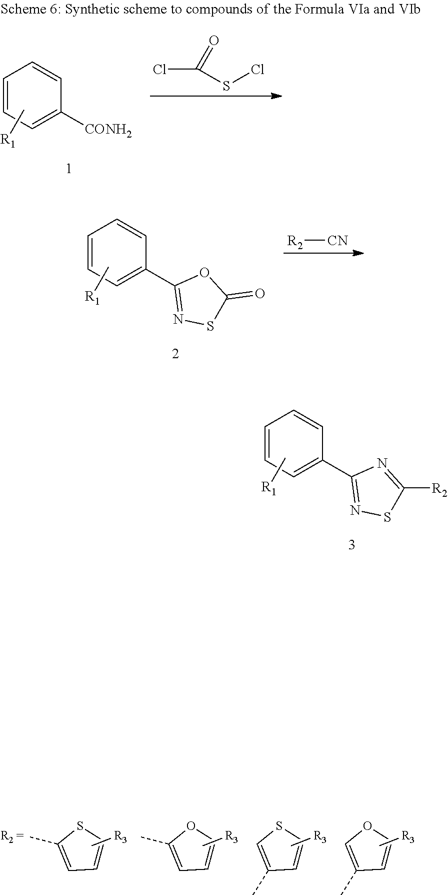

Specifically, the compounds of Formulae VIIa and VIIb can be prepared as illustrated by the exemplary reaction in Scheme 7. An appropriate furan or thiophene carboxamide substrate 1 is converted to the oxathiazolone intermediate by reacting with chlorocarbonylsulfenyl chloride. Then, the oxathiazoline intermediate 2 is reacted with an appropriate benzonitrile compound in toluene under microwave conditions to give the desired 3,5-disubstituted-1,2,4-thiadiazole product 4.

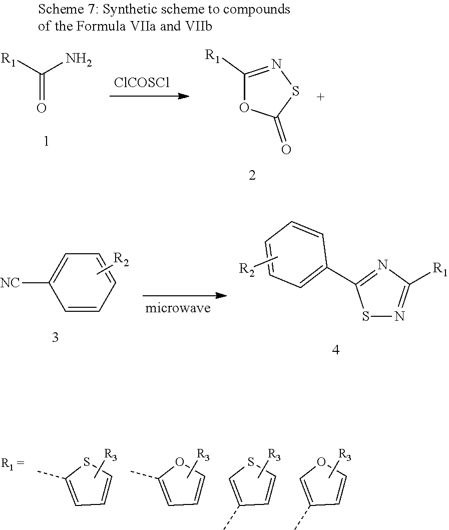

### Formula Ia Example: 5-(4-chloro-2-fluorophenyl)-2-(thiophen-2-yl)oxazole

A mixture of 4′-chloro-2′-fluoroacetophenone (17.5 g, 100 mmol), 4-(dimethylamino)pyridine tribromide (40.0 g, 110 mmol) and acetic acid (100 mL) was stirred at room temperature for 24 h. Water (150 mL) was added and after stirring for 30 min the precipitated solid was collected by filtration, washed with water, and dried in vacuo to give the desired bromide intermediate as a white solid (24 g, 95%).

To a solution of the bromide compound (24 g, 90 mmol) in acetonitrile (300 mL) was added sodium diformylamide (9.0 g, 95 mmol). The mixture was heated to reflux for 2 h and cooled to r.t overnight. The mixture was filtered to remove NaBr. The filtrate was concentrated to give diformylamide intermediate as a brown oil, 23.6 g. EtOH (300 mL) and 30% HCl (90 mL) were added and the mixture was stirred at 50° C. for 5 h and cooled to room temperature overnight, during which time the product crystallized out. The solid was collected by filtration, washed with dichloromethane, and dried to constant weight to give the desired aminoketone hydrochloride as white solid (6.3 g, 31%). that was sued as is in the next step.

The synthesis of acylamino ketone was performed as described in the literature (J. Med. Chem. 1986, 29, 333-341). A suspension of 2-amino-1-(4-chloro-2-fluorophenyl)ethanone hydrochloride (6.3 g, 28 mmol) in water (50 mL) and EtOAc (100 mL) was cooled in an ice-bath. NaHCO₃ (11.9 g, 140 mmol) was added in portions, followed by 2-thiophene carbonyl chloride (4.25 g, 29 mmol). The mixture was stirred at room temperature for 16 h. Water (50 mL) was added and the mixture was extracted with EtOAc (2×50 mL). The organic layers were combined, washed with brine, dried (MgSO₄), filtered, and concentrated in vacuo to give acylamino ketone 5 as yellow solid (7.7 g, 92%). The organic layers were combined, dried (MgSO₄), and concentrated in vacuo to give crude product, 7.8 g, which was purified by crystallization from EtOH (25 mL). Yield 5.0 g (69%) of yellow solid.

Molecular Formula: C₁₃H₇ClFNOS; MW 279.72

HPLC-ESMS: t_{R}=6.04 min; m/z: 279.9 (M+H); HPLC purity 98.0% (216 nm); 99% (250 nm)

¹H-NMR (300 MHz, CDCl₃): 7.74-7.85 (m, 2H), 7.52-7.56 (m, 1H), 7.46-7.51 (m, 1H), 7.21-7.31 (m, 2H), 7.14-7.20 (m, 1H)

### Formula IIa Example: 2-(4-chloro-2-fluorophenyl)-5-(thiophen-2-yl) oxazole

A mixture of 2-(2-bromoacetyl)thiophene (2.05 g, 10 mmol), sodium diformyl amide (1.05 g, 11 mmol) and acetonitrile (20 mL) was heated to reflux for 4 h. The mixture was cooled to r.t. and filtered to remove NaBr. The filtrate was concentrated in vacuo to give a brown oil, 2.0 g. EtOH 930 mL) was added followed by concentrated HCl (30%, 10 mL). The mixture was stirred at r.t. overnight. Concentration in vacuo gave a sticky solid, 2.1 g. The resulted aminoketone hydrochloride was contained by some NH₄Cl (based on H1-NMR spectra) and used as is in the next step.

A mixture of the crude amine.HCl in EtOAc (40 mL) and water (20 mL) was vigorously stirred and cooled in ice-water bath. NaHCO₃ (8.3 g, 100 mmol) was added, followed by 4-chloro-2-fluorobenzoyl chloride (1.9 g, 10 mmol). The mixture was stirred at r.t. overnight. The layers were separated. The water layer was extracted with EtOAc (50 mL). The combined organic layers were washed with water, dried (MgSO₄) and concentrated to a brown solid, 2.0 g. The resulted crude product was a mixture of the desired acylaminoketone and 4-chloro-2-fluorobenzamide (formed by reaction of ammonium chloride present in the starting aminoketo compound with the acyl chloride).

The acylaminoketone intermediate was dissolved n DMF (25 mL). and then POCl₃ (2.3 g, 15 mmol) was added and the mixture was stirred at r.t. for 2.5 days. Ice-water was added and the mixture was extracted with EtOAc (3×50 mL). The organic layer was washed with water (3×30 ml), dried (MgSO₄) and concentrated to a brown solid/oil, 1.7 g. A column chromatography (Hep/EtOAc 2/1) gave 1.0 g of a solid which was still not pure. Crystallization from MeOH (5 mL) gave pure (0.6 g, 22%) 2-(4-chloro-2-fluorophenyl)-5-(thiophen-2-yl)oxazole with HPLC purity >99.0% (215 and 254 nm).

Molecular Formula: C₁₃H₇ClFNOS, MW 279.72; LC-MS: t_{R}=9.46 min m/z: 279.9 (M+H).

¹H-NMR (300 MHz, CDCl₃): 7.98-8.08 (m, 1H), 7.22-7.42 (m, 5H), 7.08-7.14 (m, 1H)

### Formula IIIa Example: 2-(4-Chloro-phenyl)-5-thiophen-2-yl-[1,3,4]oxadiazole

To 250 mL round bottom flask was added 2.0 g (11.7 mmol, 1 eq) of 4-chlorobenzhydrazide (1) in 100 mL of amelene stabilized chloroform, followed by addition of 4 mL (29.25 mmol, 2.5 eq) of TEA. Then, 1.4 mL (12.87 mmol, 1.1 eq) of 2-thiophenecarbonyl chloride (2) was added drop-wise and the mixture was stirred at ambient temperature for 1 h. Reaction progress was monitored by LCMS on a twelve minute gradient. The formed white precipitate was filtered, washed with chloroform and then dried on the high vacuum for two hours. The resulting material was confirmed to be the desired diacylhydrazide and was used in the next step without further purification. The crude diacyl-hydrazide was dissolved in 60 mL of POCl₃ under heating. The resulting mixture was then heated under reflux in oil bath (100-110° C.) for 5-7 h. The reaction progress was monitored by LCMS on a twelve minute gradient. Once the cyclization reaction was completed as determined by LCMS, POCl₃ was carefully evaporated in vacuum and the reaction was then neutralized with a 1 N solution of ammonium hydroxide. The product was extracted with ethyl acetate (300 mL) from saturated solution of NaHCO₃ (200 mL), washed with a brine (2×200 mL), then dried over sodium sulfate, filtered and evaporated to dryness. The product was purified by flash column chromatography (hexane→12% ethyl acetate/hexane), and then recrystallized from mixture of hexane/ethyl acetate (5:1) to give 1.3 g of the desired compound 2-(4-Chloro-phenyl)-5-thiophen-2-yl-[1,3,4]oxadiazole (42%) as a white solid.

Chemical Formula: C₁₂H₇ClN₂OS; MW 262.71; ESMS: m/z 263 (M+H);

¹H-NMR (250 MHz, D₆-DMSO): 8.08-8.12 (m, 2H), 7.96-7.99 (m, 2H), 7.69-7.72 (m, 2H), 7.32-7.35 (m, 1H)

### Formula IVa Example: 3-(4-Chloro-2-methyl-phenyl)-5-furan-2-yl-[1,2,4-]oxadiazole

In a 500 mL round-bottom flask, 4-chloro-2-methylbenzonitrile (10 g, 66 mmol) was dissolved in 200 mL of methanol. To the mixture was added hydroxylammonium chloride (4.56 g, 66 mmol) followed by DIEA (diisopropylethylamine) (23 mL, 132 mmol). The mixture was heated at reflux for overnight. The solvents were removed. The residue was dissolved in 200 mL of CHCl₃. To the mixture was added 2-furoyl chloride (10.5 ml, 66 mmol) followed by DIEA (23 mL, 132 mmol). After reaction completion, the mixture was extracted with chloroform and water. The organic layer was separated, washed with brine, dried over Na₂SO₄, filtered and evaporated to dryness. The residue was dissolved in 200 mL of dioxanes. To the mixture was added 1 eq of DIC (N, N′-diisopropylcarbodiimide) followed by 1 eq of DIEA. The mixture was then heated at reflux overnight. After reaction completion, the mixture was cooled down. The solvents were removed in vacuo. The residue was then extracted with ethyl acetate and water. The organic layer was separated, washed with brine, dried over Na₂SO₄, filtered and evaporated to dryness. The crude was purified by flash chromatography on silica gel in a 0-20% ethyl acetate/hexanes gradient to afford 4.96 g of the desired compound 3-(4-Chloro-2-methyl-phenyl)-5-furan-2-yl-[1,2,4-]oxadiazole as a white powder in an overall yield of 28.8%.

Molecular Formula: C₁₃H₉ClN₂O₂; MW 260.04; HPLC purity 99.9% (254 nm); LC-ESMS: t_{R}=7.55 min; m/z 261.1 (M+1);

¹H-NMR (250 MHz, D₆-DMSO): 8.18-8.19 (m, 1H), 7.98-8.01 (d, J=8.3, 1H), 7.64-7.65 (m, 1H), 7.52-7.56 (m, 1H), 7.46-7.50 (m, 1H), 6.87-6.89 (m, 1H), 2.59 (s, 3H)

### Formula IVa Example: 3-(4-Bromo-2-methyl-phenyl)-5-furan-2-yl-[1,2,4]-oxadiazole

In a 500 mL round-bottom flask, 4-bromo-2-methylbenzonitrile (5 g, 25 mmol) was dissolved in 200 mL of methanol. To the mixture was added hydroxylammonium chloride (1.72 g, 25 mmol) followed by DIEA (diisopropylethylamine) (8.7 mL, 50 mmol). The mixture was heated at reflux for overnight. The solvents were removed. The residue was dissolved in 200 mL of CHCl₃. To the mixture was added 2-furoyl chloride (3.97 ml, 25 mmol) followed by DIEA (8.7 mL, 50 mmol). After reaction completion, the mixture was extracted with chloroform and water. The organic layer was separated, washed with brine, dried over Na₂SO₄, filtered and evaporated to dryness. The residue was dissolved in 200 mL of dioxanes. To the mixture was added 1 eq of DIC (N, N′-diisopropylcarbodiimide) followed by 1 eq of DIEA. The mixture was then heated at reflux overnight. After reaction completion, the mixture was cooled down. The solvents were removed in vacuo. The residue was then extracted with ethyl acetate and water. The organic layer was separated, washed with brine, dried over Na₂SO₄, filtered and evaporated to dryness. The crude was purified by flash chromatography on silica gel in a 0-20% ethyl acetate/hexanes gradient to afford 2.23 g of the desired compound 3-(4-Bromo-2-methyl-phenyl)-5-furan-2-yl-[1,2,4]-oxadiazole as a white powder in an overall yield of 36%.

Chemical Formula: C₁₃H₉BrN₂O₂; MW: 305.13; HPLC Purity >99.0%; (254 nm) ESMS: t_{R}=7.81 min; m/z 305.1 (M+¹);

¹H-NMR (250 MHz, D₆-DMSO): 8.18-8.19 (m, 1H), 7.92 (d, J=8.3, 1H), 7.58-7.70 (m, 3H), 6.86-6.90 (m, 1H), 2.59 (s, 3H)

### Formula Va Example: 5-(4-chloro-2-methylphenyl)-3-(furan-2-yl)-1,2,4-oxadiazole

To a solution of 2-furonitrile (1.9 g, 20 mmol) in MeOH (50 mL) was added hydroxylamine hydrochloride (1.4 g, 20 mmol) and triethylamine (2.1 g, 20 mmol). The mixture was heated to reflux overnight. After cooling to room temperature the mixture was concentrated in vacuo. The residue was stirred with EtOAc (50 mL). The solid was filtered off and the filtrate was concentrated to a thick oil, 2.5 g (99%). The H-NMR spectra was in accordance with the desired hydroxyamidine compound which was contaminated with Et₃N.HCl. The crude product resulted in this reaction was used without the purification in the next step.

To a suspension of 4-chloro-2-methylbenzoic acid (3.4 g, 20 mmol) in dichloromethane (50 mL) was added one drop of DMF followed by oxalylchloride (3.2 g, 25 mmol). The mixture was stirred overnight during which time all solid dissolved. The mixture was concentrated in vacuo and stripped with dichloromethane to remove excess oxalylchloride. The residual acid chloride was taken in dioxane/pyridine (10/1, 55 mL) and hydroxyamidine compound (2.5 g, 20 mmol) was added. The mixture was heated to reflux for 3 h. After cooling to room temperature, water was added (100 mL) and the resulting solid was collected by filtration and dried to give 6.2 g of crude product. Recrystallizaton from MeOH (40 mL) gave pure 5-(4-chloro-2-methylphenyl)-3-(furan-2-yl)-1,2,4-oxadiazole 2.6 g (yield 47%).

Molecular Formula: C₁₃H₉ClN₂O2; MW 260.04; HPLC purity: >99.9% (216 nm); 99.9% (324 nm); LC-ESMS: t_{R}=9.46 min; m/z 261.1 (M+1);

¹H-NMR (300 MHz, CDCl₃): 8.10 (dd, J=8.1, 1H), 7.63-7.66 (m, 1H), 7.32-7.42 (m, 2H), 7.18-7.22 (d d, J=2.7, 0.9, 1H), 6.58-6.62 (m, 1H), 2.89 (s, 3H)

### Formula VIa Example: (2,4-dichlorophenyl)-5-(furan-2-yl)-1,2,4-thiadiazole

A mixture of 2,4-dichlorobenzamide (25 g, 131.5 mmol) and chlorocarbonylsulfenylchloride (19 g, 145 mmol) in toluene (150 mL) was heated to reflux for 4 h (HCl-gas formation was observed with pH paper). After cooling to r.t. the mixture was concentrated in vacuo to give the desired oxathiazolone compound as an off-white solid (32.4 g, 99%). that was used in the next without purification. In a 20 mL vial a mixture of oxathiazolone 8a (2 g, 8 mmol) and 2-furonitrile (10 g, 107 mmol) was heated in the microwave at 190° C. for 20 min. The reaction was performed 10 times and the combined mixture was distilled (Kugerrohr) at 100° C./20 mbar to remove excess 2-furonitrile (the recovered 2-furonitrile was used again). The mixture was further distilled at 150° C./10 mbar to remove the byproduct nitrile 10 (yellow solid, 6.5 g, 47%). The residue of the distillation (circa 10 g) was taken in dichloromethane (50 mL), filtered and the filtrate concentrated to a brown solid, 8 g. Recrystallization by dissolution in hot MeOH (50 mL) and addition of water (10 mL) gave pure (2,4-dichlorophenyl)-5-(furan-2-yl)-1,2,4-thiadiazole as brown solid, 4.7 g, in a 20% yield.

Chemical Formula: C₁₂H₆Cl₂N₂OS; MW: 297.16; HPLC-ESMS: t_{R}=6.5; m/z: 296.96; 298.95 (M+1); HPLC purity >99% (221 nm),>99% (263 nm), >99.0% (306 nm)

¹H-NMR (300 MHz, CDCl₃): 7.90 (dd, J=8.4, 1H), 7.57-7.58 (m, 1H), 7.29 (dd, J=8.4, 1.8) 7.48, d, J=1.8, 1H), 7.15-7.20 (m, 1H), 6.55-6.59 (m, 1H)

### Formula VIa Example: 3-(4-chloro-2-methylphenyl)-5-(furan-2-yl)-1,2,4-thiadiazole

A magnetically stirred mixture of acid 4-chloro-2-methylbenzoic acid (50 g, 0.29 mol), dichloromethane (200 mL), and 0.5 mL DMF was cooled in an ice-bath. The cooler was connected to a gas absorption trap. Oxalyl chloride (44.5 g, 0.35 mmol) was added dropwise in 1 h. The mixture was stirred at r.t. overnight during which time all solid dissolved. The solution was concentrated in vacuo and stripped with dichloromethane to remove excess oxalyl chloride. The residue was taken in THF (200 mL) and mechanically stirred in an ice-water bath. Aqueous 25% ammonia (100 mL) was added in 15 min, which resulted in the formation of a precipitate. The THF was removed with the rotavap and extra water (100 mL) was added. The suspension was stirred at r.t. overnight. The solid was collected by filtration and dried in vacuo to give 2-methyl-4-chlorobenzamide (43.7 g, yield 89%) that was used without purification in the next step.

A mechanically stirred mixture of 2-methyl-4-chlorobenzamide (31.35 g, 185 mmol), toluene (400 mL), and chlorocarbonylsulfenylchloride (25 g, 190 mmol) was heated to reflux for 3 h. After cooling to room temperature the mixture was concentrated in vacuo to give a yellow solid 40 g (95%). H-NMR showed that this was a mixture of the desired oxathiazolone compound and nitrile by-product and starting amide in a ratio 85:10:5. This mixture was used in the next step without further purification.

The crude oxathiazolone compound (2.0 g, 8.8 mmol) and 2-furonitrile (16 g, 170 mmol) were mixed and heated for 20 min at 190° C. in the microwave. Ten batches were combined and Kugelrohr distilled at 100° C./30 mbar to recover excess 2-furonitrile (used again in next microwave reactions). The residue was further distilled at 150° C./20 mbar to remove the nitrile by-product. The residue, 5.5 g was combined with the residue of another ten microwave reactions (4.5 g) and purified by column chromatography. The resulting 4.5 g (85% pure by HPLC) was recrystallized from MeOH (50 mL) to give pure 3-(4-chloro-2-methylphenyl)-5-(furan-2-yl)-1,2,4-thiadiazole as light brown solid, 3.6 g (7.5% yield).

Chemical Formula: C₁₃H₉ClN₂OS; MW: 278.7; HPLC-ESMS: t_{R}=6.36 min and m/z 277.0 (M+1); HPLC purity: >95% (220 nm) 95% (270 nm).

¹H-NMR (300 MHz, CDCl₃): 8.06, (dd, J=7.8, 1H), 7.62-7.63 (m, 1H), 7.22-7.31 (m, 3H), 6.61-6.63 (m, 1H), 2.66 (s, 3H)

### Formula VIa Example: 3-(4-chlorophenyl)-5-(furan-2-yl)-1,2,4-thiadiazole

A mechanically stirred mixture of 4-chlorobenzamide (20.23 g, 130 mmol), toluene (150 mL), and chlorocarbonylsulfenylchloride (19 g, 145 mmol) was heated to reflux for 3 h. After cooling to r.t. the mixture was concentrated in vacuo to give a yellow solid foam, 27.65 g (100%). H-NMR showed that this was almost pure oxathiazolone compound that was used as is in the next step. The oxathiazolone compound (1.71 g, 8 mmol) and 2-furonitrile (15 g, 160 mmol) were mixed and heated for 20 min at 190° C. in the microwave. Ten batches were combined and Kugelrohr distilled at 100° C./30 mbar to recover excess 2-furonitrile (used again in next microwave reactions). The residue was further distilled at 150° C./20 mbar to remove the nitrile byproduct. The residue, 5 g, was recrystallized from MeOH to give 3.5 g of solid. This was combined with the residue of another 5 microwave reactions (2.6 g) and purified by column chromatography. The resulting 4.4 g (90% pure by HPLC) was recrystallized from Heptane/EtOAc=7/1 (50 mL) to give pure 3-(4-chlorophenyl)-5-(furan-2-yl)-1,2,4-thiadiazole as light brown solid, 3.35 g (10% yield).

Chemical Formula: C₁₂H₇ClN₂OS; Molecular Weight: 262.71; HPLC-ESMS: t_{R}=6.06 min; m/z: 263.00, 264.99 (M+1)

¹H-NMR (300 MHz, CDCl₃): 8.24-8.33 (m, 2H), 7.63-7.65 (m, 1H), 7.42-7.50 (m, 2H), 7.23-7.28 (m, 1H), 6.62-64 (m, 1H)

### Formula VIIa Example: 5-(2-chloro-4-methylphenyl)-3-(furan-2-yl)-1,2,4-thiadiazole

A magnetically stirred mixture of 2-furoylamide (prepared from 2-furoylchloride and aqueous ammonia, 1.13 g, 10 mmol) and chlorocarbonylsulfenylchloride (2.0 g, 15 mol) in toluene (20 mL) was heated to reflux for 4 h. After cooling to room temperature the mixture was concentrated to give 1.7 g of the desired oxathiazolone as a yellow solid (almost in a quantitative yield) that was used in the next step without further purification.

A mixture of the oxathiazolone compound (170 mg, 1 mmol) and 4-chloro-2-methylbenzonitrile (3.03 g, 20 mmol) was heated in the microwave at 190° C. for 20 min. A second reaction was performed and the mixtures were combined. Excess of the nitrile by-product (furonitrile) were removed in vacuo (120° C., 0.3 mbar). The residual brown solid (100 mg) was taken in hot MeOH (10 mL) and decanted from insoluble material (presumably sulphur). The MeOH solution was left at room temperature overnight. The precipitated solid was collected and dried to give compound 5-(2-chloro-4-methylphenyl)-3-(furan-2-yl)-1,2,4-thiadiazole as brown solid, 40 mg (7%). NMR conform structure.

Chemical Formula: C₁₃H₉ClN₂OS; MW: 278.7; HPLC-ESMS: t_{R}=6.36 min and m/z 277.01 (M+1); HPLC purity: 93.5 (216 nm) 91% (324 nm); ¹H-NMR (300 MHz, CDCl₃): 7.87 (dd, J=8.1, 1H), 7.51-7.60 (m, 1H), 7.24-7.32 (m, 2H), 7.15-7.20 (m, 1H), 6.50-6.56 (m, 1H), 2.58 (s, 3H)

### Example 2: Field Trials

A field trial was conducted to determine the efficacy of compounds as described herein against soybean sudden death syndrome. Soybean seeds were planted at the start of the spring growing season in a location where fungi of the genus Fusarium were endemic. Nothing special was done to encourage growth of the fungus or development of the SDS disease.

Compounds listed in Table 1 below were used to treat soybean seeds prior to planting.

TABLE 1

| Name | Structure |
|---|---|
| DC1822 | 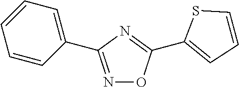 [SMILES: c1ccc(-c2noc(-c3cccs3)n2)cc1] |
| DC5523 | 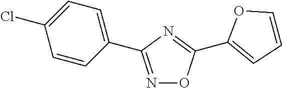 [SMILES: Clc1ccc(-c2noc(-c3ccco3)n2)cc1] |
| DC5823 | 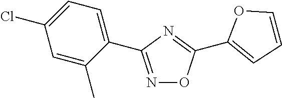 [SMILES: Cc1cc(Cl)ccc1-c1noc(-c2ccco2)n1] |

Seeds treated with oxamyl, at a rate of 1 mg per seed, and non-treated seeds (NT) were included as controls.

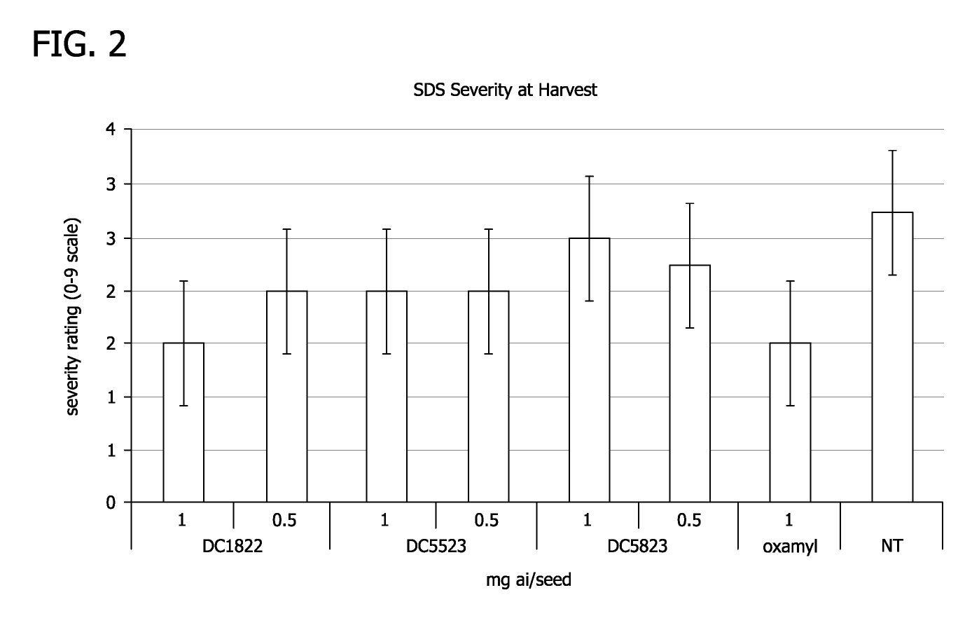

At harvest time in the fall, all plots in the experiment were evaluated for the incidence of plants exhibiting SDS symptoms, as well as the average severity of symptoms in the plots. Other typical agronomic data, including counts of soybean cyst nematode (SCN) eggs, were also collected throughout the trial. The results of the experiments are shown in Table 2 below. The percentage of affected plants and the observed SDS disease severity are graphically depicted in FIGS. 1 and 2, respectively.

The results indicate that treatment of soybean seeds with compounds and compositions as described herein can provide significant control of SDS.

TABLE 2

| | mg/ | Spring Eggs/ | Fall Eggs/ | Mean | SDS | SDS |
|---|---|---|---|---|---|---|
| Treatment | seed | 100 cc | 100 cc | RF | incidence | severity |
| DC1822 | 1 | 700 | 2100 | 6.425 | 28.75 | 1.5 |
| DC1822 | 0.5 | 1175 | 2600 | 3.875 | 45 | 2 |
| DC5523 | 1 | 800 | 1725 | 4.325 | 26.25 | 2 |
| DC5523 | 0.5 | 675 | 1925 | 5.35 | 47.5 | 2 |
| DC5823 | 1 | 1450 | 1100 | 1.625 | 46.25 | 2.5 |
| DC5823 | 0.5 | 1075 | 3325 | 3.35 | 55 | 2.25 |
| Oxamyl | 1 | 1050 | 3075 | 6.65 | 32.5 | 1.5 |
| NT | | 750 | 2525 | 16.575 | 43.75 | 2.75 |

When introducing elements of the disclosed methods, compounds, or compositions or the preferred embodiments(s) thereof, the articles “a”, “an”, “the” and “said” are intended to mean that there are one or more of the elements. The terms “comprising”, “including” and “having” are intended to be inclusive and mean that there can be additional elements other than the listed elements.

In view of the above, it will be seen that the several objects are achieved and other advantageous results attained.

As various changes could be made in the above products and methods without departing from the scope of the disclosure, it is intended that all matter contained in the above description and shown in the accompanying drawings shall be interpreted as illustrative and not in a limiting sense.

1. A method of controlling a root-originated disease in a plant located in a field with nematode pressure, the method comprising administering to a plant, a seed, or soil a composition comprising an effective amount of a compound of Formula IV, Formula V, or a salt thereof 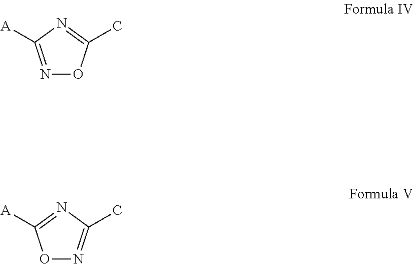
wherein
A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and
C is selected from the group consisting of thienyl, furanyl, oxazolyl, and isoxazolyl, each of which can be optionally independently substituted with one or more substituents selected from fluorine, chlorine, CH₃, and OCF₃.

2. (canceled)

3. The method of claim 1 wherein the root-originated disease is caused by Fusarium, Pythium, Phytopthora, or Rhizoctonia.

4. The method of claim 1 wherein the root-originated disease is selected from the group consisting of Fusarium root rot, Fusarium wilt, sudden death syndrome, corn stalk rot, seedling blight, charcoal rot, and Verticillum wilt.

5. A method of controlling soybean sudden death syndrome in a plant located in a field with nematode pressure, the method comprising administering to a soybean plant, a soybean seed, or soil a composition comprising an effective amount of a compound of Formula IV, Formula V, or a salt thereof 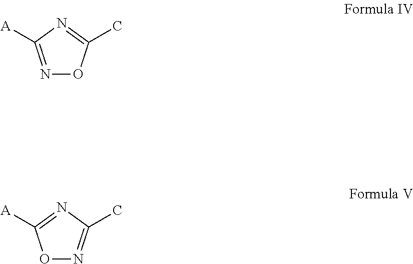
wherein
A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and
C is selected from the group consisting of thienyl, furanyl, oxazolyl, and isoxazolyl, each of which can be optionally independently substituted with one or more substituents selected from fluorine, chlorine, CH₃, and OCF₃.

6. The method of claim 1 wherein the composition is administered to a soybean plant, a soybean seed, or soil in a location wherein soybean plants are at a significant risk of disease caused by fungi of the genus Fusarium.

7. The method of claim 6 wherein the composition is administered to a soybean plant, a soybean seed, or soil in a location wherein soybean plants are at a significant risk of soybean sudden death syndrome.

8. A method of improving the yield of a soybean plant grown from a seed, the method comprising planting a soybean seed comprising a composition comprising an effective amount of a compound of Formula IV, Formula V, or a salt thereof 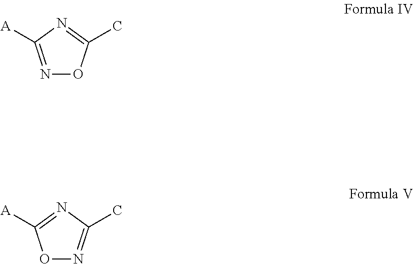
wherein
A is selected from the group consisting of phenyl, pyrazyl, oxazolyl, and isoxazolyl, each of which can be optionally independently substituted with one or more substituents selected from halogen, CF₃, CH₃, OCF₃, OCH₃, CN, and C(H)O; and
C is selected from the group consisting of thienyl, furanyl, oxazolyl, and isoxazolyl, each of which can be optionally independently substituted with one or more substituents selected from fluorine, chlorine, CH₃, and OCF₃,
and wherein the soybean seed is planted in a location with nematode pressure and wherein soybean plants are at a significant risk of disease caused by fungi of the genus Fusarium.

9. The method of claim 8 wherein the soybean seed is planted in a location wherein soybean plants are at a significant risk of soybean sudden death syndrome.

10. The method of claim 1 wherein A is phenyl.

11.-13. (canceled)

14. The method of claim 1 wherein C is thienyl.

15. The method of claim 1 wherein C is furanyl.

16.-17. (canceled)

18. The method of claim 1 wherein the composition comprises a compound of Formula IVa or a salt thereof, 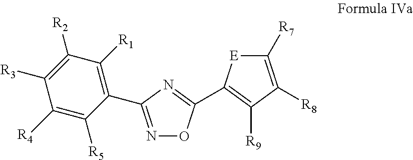
wherein R₁ and R₅ are independently selected from hydrogen, CH₃, F, Cl, Br, CF₃ and OCF₃;
R₂ and R₄ are independently selected from hydrogen, F, Cl, Br, and CF₃;
R₃ is selected from hydrogen, CH₃, CF₃, F, Cl, Br, OCF₃, OCH₃, CN, and C(H)O;
R₇ and R₈ are independently selected from hydrogen and fluorine;
R₉ is selected from hydrogen, F, Cl, CH₃, and OCF₃; and
E is O or S.

19. The method of claim 1 wherein the composition comprises a compound of Formula IVb or a salt thereof, 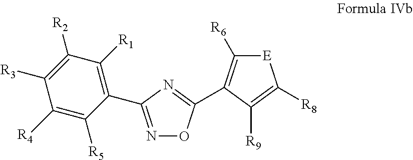
wherein R₁ and R₅ are independently selected from hydrogen, CH₃, F, Cl, Br, CF₃ and OCF₃;
R₂ and R₄ are independently selected from hydrogen, F, Cl, Br, and CF₃;
R₃ is selected from hydrogen, CH₃, CF₃, F, Cl, Br, OCF₃, OCH₃, CN, and C(H)O;
R₈ is selected from hydrogen and fluorine;
R₆ and R₉ are independently selected from hydrogen, F, Cl, CH₃, and OCF₃; and
E is O or S.

20. The method of claim 1 wherein the composition comprises a compound of Formula Va or a salt thereof, 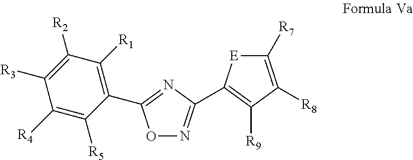
wherein, R₁ and R₅ are independently selected from hydrogen, CH₃, F, Cl, Br, CF₃ and OCF₃;
R₂ and R₄ are independently selected from hydrogen, F, Cl, Br, and CF₃;
R₃ is selected from hydrogen, CH₃, CF₃, F, Cl, Br, OCF₃, OCH₃, CN, and C(H)O;
R₇ and R₈ are independently selected from hydrogen and fluorine;
R₉ is selected from hydrogen, F, Cl, CH₃, and OCF₃;
and E is O or S.

21. The method of claim 1 wherein the composition comprises a compound of Formula Vb or a salt thereof, 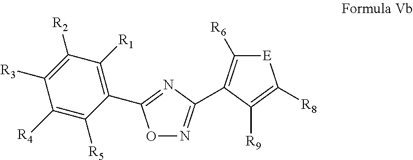
wherein R₁ and R₅ are independently selected from hydrogen, CH₃, F, Cl, Br, CF₃ and OCF₃;
R₂ and R₄ are independently selected from hydrogen, F, Cl, Br, and CF₃;
R₃ is selected from hydrogen, CH₃, CF₃, F, Cl, Br, OCF₃, OCH₃, CN, and C(H)O;
R₈ is selected from hydrogen and fluorine;
R₆ and R₉ are independently selected from hydrogen, F, Cl, CH₃, and OCF₃; and
E is O or S.

22. The method of claim 18 wherein E is O.

23. The method of claim 18 wherein E is S.

24. The method of claim 1 wherein the composition comprises a compound selected from the group consisting of:
3-phenyl-5-(thiophen-2-yl)-1,2,4-oxadiazole,
5-(furan-2-yl)-3-phenyl-1,2,4-oxadiazole,
3-(4-fluorophenyl)-5-(thiophen-2-yl)-1,2,4-oxadiazole,
3-(4-fluorophenyl)-5-(furan-2-yl)-1,2,4-oxadiazole,
3-(4-chlorophenyl)-5-(thiophen-2-yl)-1,2,4-oxadiazole,
3-(4-chlorophenyl)-5-(furan-2-yl)-1,2,4-oxadiazole,
3-(4-bromophenyl)-5-(thiophen-2-yl)-1,2,4-oxadiazole,
3-(4-bromophenyl)-5-(furan-2-yl)-1,2,4-oxadiazole,
3-(4-chloro-2-methylphenyl)-5-(furan-2-yl)-1,2,4-oxadiazole,
3-(2,4-dichlorophenyl)-5-(furan-2-yl)-1,2,4-oxadiazole,
3-(4-chlorophenyl)-5-(thiophen-3-yl)-1,2,4-oxadiazole,
3-(4-chlorophenyl)-5-(furan-3-yl)-1,2,4-oxadiazole,
3-(4-fluorophenyl)-5-(thiophen-3-yl)-1,2,4-oxadiazole, and
3-(4-fluorophenyl)-5-(furan-3-yl)-1,2,4-oxadiazole.

25. The method of claim 24 wherein the composition comprises 3-phenyl-5-(thiophen-2-yl)-1,2,4-oxadiazole.

26. The method of claim 1 wherein the composition further comprises one or more components selected from the group consisting of an insecticide, a fungicide, an herbicide, a nematicide, an aqueous surfactant, and a co-solvent.
